SEQUENCE LISTING (48 sequences)

SEQ ID NO: 1 - DNA, 372 bp, Zea mays
atggccactcaaagtccccatgttctcgttgtggatcattcccgtgttgattgccttgttgcatcgattgtcctcaatagtttcaacattcgagtaactgttgcgggaggtgccatggaagcattggaattcttagatgcgaacaacagtcatgtggaccttattttgactgactactgcatgcctgatatgactggctatgatttgcttagggaagtgaaggaatcgccaagactaaagcacattccagtggtgattacatgcactgatgtcataccagaaagaatcattgagtgctttgaaggaggagcagaggaatacatgataaagcctctcaaggtttctgacgtgcctcgtattcttagctacatg

SEQ ID NO: 2 - protein, 124 aa, Zea mays
MATQSPHVLVVDHSRVDCLVASIVLNSFNIRVTVAGGAMEALEFLDANNSHVDLILTDYCMPDMTGYDLLREVKESPRLKHIPVVITCTDVIPERIIECFEGGAEEYMIKPLKVSDVPRILSYM

SEQ ID NO: 3 - DNA, 216 bp, Zea mays
atggccactcaaagtccccatgttctcgttgtggatcattcccgtgttgattgccttgttgcatcgattgtcctcaatagtttcaacattcgaggtatcatggttatcatgagtttttataataactatcgagtagtttttacgaatgttctaatattatcattttcatattttttataaaaatagagaacattggtacaatattatcttagatat

SEQ ID NO: 4 - DNA, 215 bp, Zea mays
  misc_feature (8)..(8): n is a, c, g, or t
atggccantcaaagtccccatgttctcgttgtggatcattcccgtgttgattgccttgttgcatcgattgtcctcaatagtttcaacattcgaggtatcatgattatcatgagtttttataataactatcaagtagtttttacgaatgttctaatattatcattttcatatatttttataaaaatagagaacgttggtacaatattatctgagat

SEQ ID NO: 5 - DNA, 215 bp, Zea mays
  misc_feature (208)..(208): n is a, c, g, or t
atggccactcaaagtccccatgttctcgttgtggatcattcccgtgttgattgccttgttgcatcgattgtcctcaatagtttcaacattcgaggtatcatggttatcatgagtttttataataactatcgagtagtttttacgaatgttctaatattatcattttcatattttttataaaaatagagaacattggtacaatattatnttagaat

SEQ ID NO: 6 - DNA, 216 bp, Zea mays
  misc_feature (210)..(210): n is a, c, g, or t
atggccactcaaagtccccatgttctcgttgtggatcattcccgtgttgattgccttgttgcatcgattgtcctcaatagtttcaacattcgaggtatcatggttatcatgagtttttataataactatcgagtagtttttacgaatgttctaatattatccattttcatatatttttataaaaatagagaacgttggtacaatattatnttagaa

SEQ ID NO: 7 - DNA, 216 bp, Zea diploperennis
  misc_feature (209)..(209): n is a, c, g, or t
atggccactcaaagtccccatgttcttgttgtggatcattcccgtgttgattgccttgttgcatcgattgtcctcaatagtttcaacattcgaggtatcatggttatcatgagtttttataataactatcgagtagtttttatgaatgttctaatattatcattttcatatatttttataaaaatagagaatgttggtacaatattatntcagaat

SEQ ID NO: 8 - DNA, 1187 bp, Zea mays
aagcttagcttcataggatgatccactaaggttgcataattttatagatcataagatgctcacaaggtcattagggatatcttgcttctacatgaagaacaccataatcagatagttcactattgtttaaccatatcgtctacttcttgctctagatcagtttttcatcttgtttcaaatttgatctcaaccaatccaacatctttgatatccttgacttcaaaccatactaataaggacacttacgatccttaacttgattaagagttagccacttagtgcagcacaatgctctctcttcatctctttcaaggtgaatgccttagatcctccttagtagtatagataaaggaaaataaagactatattactacttagcaaaaatccccatggtcgtatcctacattctattttagtgttttctgaggcatcatgaatatactttgcaccatacgtagcccactaacaaagtctagggaagtagataagtggtcactcacattgtgactaattcaaatgcattaaccaagtagaaaccactttagccaaaactacttgttgtcctaaatcattgaaatgatgaaaccccatagtctccacagatggtgagtataacatggaattcaaattctattgatacttcttatatgaagaattgaatatttttattccatagtgcatgacaattagatacaacaagtatttatttttttatctcaaagatatcaatataaatccacaattactatgtaaatatgtgtgtcttggtcttagtctcccatggtcattttccctcctatatgccattattttttatttttttattgctagataatatcgattggatctttaggaatagatatgtagatctaatgggcacaaatatgcaatatatattcaattttcacatttcaagatatggtagttttttctggctactgctaagtgatagatcatcttatcgcgtcaagagatatgatagccaaatccagctactatttcatatccccgtatcttaagcatatcattatttctctatcttaagcatatcgctagtttcttagggatattttagttgtccatggtttactatataaacaagtctattccaccttcagtggcacaaaacattgtaacaaactacatatactaaagtttagtacattgagagcttgtctagctagtcc

SEQ ID NO: 9 - DNA, 2419 bp, Zea mays
  misc_feature (2407)..(2407): n is a, c, g, or t
aagcttagcttcataggatgatccactaaggttgcataattttatagatcataagatgctcacaaggtcattagggatatcttgcttctacatgaagaacaccataatcagatagttcactattgtttaaccatatcgtctacttcttgctctagatcagtttttcatcttgtttcaaatttgatctcaaccaatccaacatctttgatatccttgacttcaaaccatactaataaggacacttacgatccttaacttgattaagagttagccacttagtgcagcacaatgctctctcttcatctctttcaaggtgaatgccttagatcctccttagtagtatagataaaggaaaataaagactatattactacttagcaaaaatccccatggtcgtatcctacattctattttagtgttttctgaggcatcatgaatatactttgcaccatacgtagcccactaacaaagtctagggaagtagataagtggtcactcacattgtgactaattcaaatgcattaaccaagtagaaaccactttagccaaaactacttgttgtcctaaatcattgaaatgatgaaaccccatagtctccacagatggtgagtataacatggaattcaaattctattgatacttcttatatgaagaattgaatatttttattccatagtgcatgacaattagatacaacaagtatttatttttttatctcaaagatatcaatataaatccacaattactatgtaaatatgtgtgtcttggtcttagtctcccatggtcattttccctcctatatgccattattttttatttttttattgctagataatatcgattggatctttaggaatagatatgtagatctaatgggcacaaatatgcaatatatattcaattttcacatttcaagatatggtagttttttctggctactgctaagtgatagatcatcttatcgcgtcaagagatatgatagccaaatccagctactatttcatatccccgtatcttaagcatatcattatttctctatcttaagcatatcgctagtttcttagggatattttagttgtccatggtttactatataaacaagtctattccaccttcagtggcacaaaacattgtaacaaactacatatactaaagtttagtacattgagagcttgtctagctagtccatggccactcaaagtccccatgttctcgttgtggatcattcccgtgttgattgccttgttgcatcgattgtcctcaatagtttcaacattcgaggtatcatggttatcatgagtttttataataactatcgagtagtttttacgaatgttctaatattatcattttcatattttttataaaaatagagaacattggtacaatattatcttagatatgatatcgacacttgaatccgtgcaattatctacaccttgaactacaccataaatacaagtttgtactatgttatccaacatctatatagtagagaaaacatgagaattatgcaattttattttactatgatccattgttctttgtgtgtaaatgtgttttttcttcagtaactgttgcgggaggtgccatggaagcattggaattcttagatgcggtactcatcaatattttgtatatctatgtacttctttaagttattgtgattgtttactatgtttgacttgtttacatcacgatcttggaacatatagaacaacagtcatgtggaccttattttgactgactactgcatgcctgatatgactggctatgatttgcttagggaagtgaaggtaaatcctgttttggccattataattttattcaaagcttaatctcatagtattttagtttaatattattataatgcttcataatattctacaacacaaataatgcataacaaactttttccaatctaggaatcgccaagactaaagcacattccagtggtgattacatgcactgatgtcataccagaaagaatcattgagtgagtaaaggaaaaacaatgaaatattgcatgagtgaaacatatgttatctaagtaaccaaaatatttatgattttccttattcaggtgctttgaaggaggagcagaggaatacatgataaagcctctcaaggtttctgacgtgcctcgtattcttagctacatgtgatgcgcttgagggagaaaacgaataaaaaggaaatgcaagcataaacagtctttgggttatagttctctatcaatcatgcttaagtgtgaccatatatggtcttttattatgtcacctgttgttttgaataatttgtagccaatgcaagcctaatcagtatttggcttatagttctctatcaatcatgcttatgtgtggccatctatggtacttttaatgtcatccattattttttttcaaancacaaaanacac

SEQ ID NO: 10 - DNA, 24 bp, Artificial
  Primer RRfw
ctagtccatggccactcaaagtcc

SEQ ID NO: 11 - DNA, 26 bp, Artificial
  Primer 3810rw
aggcttgcattggctacaaattattc

SEQ ID NO: 12 - DNA, 23 bp, artificial
  Primer RR1
gcacggattcaagtgtcgatatc

SEQ ID NO: 13 - DNA, 28 bp, artificial
  Primer RR2
tgcggtactcatcaatattttgtatatc

SEQ ID NO: 14 - DNA, 26 bp, Artificial
  Primer RR3
aaactcatgataaccatgatacctcg

SEQ ID NO: 15 - DNA, 28 bp, Artificial
  Primer RR4
cgatcttggaacatatagaacaacagtc

SEQ ID NO: 16 - DNA, 31 bp, artificial
  pBETL9fw
cgatggtacttactcatgatggtcatctagg

SEQ ID NO: 17 - DNA, 27 bp, artificial
  pBETL9rw
ccatggtataacttcaactgttgacgg

SEQ ID NO: 18 - DNA, 1911 bp, Zea mays
ctcgagttactcatgatggtcatctaggatcatagaccatccccacagaccaacatgagtcttttctacgcactttgttcactcgtgtgcatcaaagaaaacttcttggttggtcactcatccaaaaattgctctgagccaagcatgcttatcttagaggtttttttgagataagcttctgaaaaagaaggtgcaccttgtttgtatgagtattataccattcctattaagccttggacaaagatatcacaatccacctaggccaagatatcacatttcccaacttagtctataaaaggactagacaagacatctccttagaagagaagccctacctcttgtgcccataacaggcacctccaacttgagaactaatttcacaaagagtcacgctcttgggaactccatgtactcatatgtacacactacatctaatgcatagaaacaccaagatcacattgtactagcaaaatgtcatagaagactagttaaaaccttgtttggtctgctcaaacttaacaaatcacctaggagacatgctagaagtatctcaacagagaatgacaccatatagtagtggcaccaagtgcctaatctgcacacaaaaaaatcgtaccatacatgacatcaaggcttaataatagagtgtatgttaaagcgagcatgcaacctatgagtggtatgtaggagttaggtttaaacaaggtaatggctcaagcaccacacatcctaccacaatgtcgtaataaatataaaagcactagcaatctatttagcatgcctaaatgggatactatgaggttgggtgggatgtggcacctttgtataatggcccagttccttagtgtagtcttgatcctccccgttatgttgagactcctctagggattttgtaggaatcatcaaattttcataagcaatttcttgtgcacaaagaaccaaatagattgaaaaagtttcaaattcactcaaacacaaaaccatggcacatagcttatgtgacaaaatatttgggacactagtttcatattttttgagatcatataagtttattatcaaactcccaaggattaaattattttttgaaaaaaaagaaaaaagggaaaacatcataaggtgacacatggcaacctctgaatgactagacttttaccatctctcaggtgggtctggtcaacaatcactgttggtcggtccttaccttgcctagacgggtccttagtaggcctactgggttgagttatgggataaattgtggcctagaaacataccagtccaccaaccttgggaccacttaaaaaattgcatctttcaccattatactatttagatgtttttaaaaaacaatcataacttttacatcgaaatcaaaactagacaaattttatactttcacagagcagcagaaatttatacaatatgattgaatacaagatgtaggacccaatggagagaattttttgtctcctatatgcttgaatacccaacataatatcttcgcaacatactatctatctaatagaaaaattataatatagttaaatacttaagtagtatctagtggatagaattcaatatctcctacatgcatgaggagtaatatctactagacatgcaacatatttttatctatctaatagaatatatataataaagttaaatattatatgcatcacctactatatataatttgatatcttttagatgtataagggactaagaataatatctctagcacacatgcaatgcattatctatctaaatatattatataatagttaaatattaattatacgtagtctaaacctacatataagcctacccatccccacttaaagatctcagtgtcacacatagaccatacatctcacttcgccaaaaaaattccgtcaacagttgaagttataccct

SEQ ID NO: 19 - protein, 153 aa, Zea mays
MAAAAPAPASVAPSSAPKATGDSRKTVVSVDASELEKHVLAVDDSSVDRAVIARILRGSRYRVTAVESATRALELLALLPDVSMIITDYWMPGMTGYELLKCVKESAALRGIPVVIMSSENVPTRITRCLEEGAEGFLLKPVRPADVLCSRIR

SEQ ID NO: 20 - protein, 138 aa, Zea mays
MAAAEARGGEFPVGMKVLVVDDDPTCLVVLKRMLLECRYDVTTCPQATRALTMLRENRRGFDVIISDVHMPDMDGFRLLELVGLEMDLPVIMMSADSRTDIVMNGVKHGACDYLIKPVRMEELKNIWQHVIRKKFNEN

SEQ ID NO: 21 - protein, 157 aa, Zea mays
MAAAATATPSVAPESGDRKAVAPPVDAVDLELELEEKHVLAVDDSSVDRAVIAKILRSSKYRVTTVDSATRALELLALGLVPDVNMIITDYWMPGMTGYELLKHVKESSALRAIPVVIMSSENVPTRISRCLEEGAEDFLLKPVRPADVSRLCSRIR

SEQ ID NO: 22 - protein, 135 aa, Zea mays
MASRKCLGGEGSAPAPHVLAVDDSSVDRAVIAGILRSSQFRVTAVDSGKRALELLGTEPNVSMIITDYWMPEMTGYELLKKIKESSRLKEIPVVIMSSENVPTRINRCLEEGAEDFLLKPVRPSDVSRLCSRVLR

SEQ ID NO: 23 - protein, 161 aa, Zea mays
MTVLDAESRFHVLAVDDSIIDRKLIEMLLKSSSYQVTTVESGNKALELLGLRDNGAEDASPPSSSSSSSSSSSPDHQEIDVSLIITDYCMPGMTGYDLLKRVKGSSSLKDIPVVIMSSENVPARISRCLQDGAEEFFLKPVKPADMKKLKSHLLKRKQPKQ

SEQ ID NO: 24 - protein, 149 aa, Zea mays
MTVPDAESRFHVLAVDDSLVDRKLIEMLLKTSSYQVTTVDSGSKALELLGLRDASSPSPSSPDHQEIDVNLIITDYCMPGMTGYDLLKRVKGSSSLKDIPVVIMSSENVPARISRCLQDGAEEFFLKPVKLADMKKLKSHLLKRKQPKE

SEQ ID NO: 25 - protein, 187 aa, Zea mays
MPSQSTHTSSSLSSSTAPILPCSSAAALFRSVMAAVATETPFHVLAVDDSLPDRKLIERLLKTSSFQVTTVDSGSKALQFLGLDQDSTVPPVHTHQLDVAANQDVAVNLIITDYCMPGMTGYDLLKKIKESSSLRDIPVVIMSSENIPSRINRCLEEGADEFFLKPVRLSDMNKLKPHILKSRCNQE

SEQ ID NO: 26 - protein, 160 aa, Zea mays
MGQGGEVKAAPAVRVLVVDDSPVDRKVVELLLRNHNHQGGAAPFHVTAVDSGKKAMEHLRLMEQGGQLDSCAADANRITIDIVLTDYCMPEMTGYDLLKAIKALSSPNPIPVVVMSSENEPQRISRCLTAGAEDFILKPLKTKDVQRLRNCSSAARPRDD

SEQ ID NO: 27 - protein, 165 aa, Zea mays
MAAAEARGADFPVGMKVLVVDDDPTCLVVLKRMLLECRYDVTTCPQATRALTMLRRRGFDVIISDVHMPDMDGFRLLELVGLEMDLPVIMMSADSRTDIVMKGIKHGACDYLIKPVRMEELIWQHVVRKKFNGNKDHEHSGSLDDTDRNRPTNNDNEYASSANDG

SEQ ID NO: 28 - protein, 149 aa, Zea mays
MTVDELKLQARASGGHGAKDQFPVGMRVLAVDDDPTCLKILENLLLRCQYHVTTTGQAATALKLLREKKDQFDLVISDVHMPDMDGFKLLELVGLEMDLPVIMLSANGETQTVMKGITHGACDYLLKPVRIEQLRTIWQHVVRRRSCDA

SEQ ID NO: 29 - protein, 122 aa, Zea mays
MATQSPHVLVVDHSRVDCLVASIVLNSFNIRVTVAGGAMEALEFLDNSHVDLILTDYCMPDMTGYDLLREVKESPRLKHIPVVITCTDVIPERIIECFEGGAEEYMIKPLKVSDVPRILSYM

SEQ ID NO: 30 - protein, 8 aa, Artificial
  synthetic consensus sequence
HVLAVDDS

SEQ ID NO: 31 - protein, 16 aa, Artificial
  Synthetic consensus sequence
VTTVDSATRALELLGL

SEQ ID NO: 32 - protein, 6 aa, Artificial
  synthetic consensus sequence
LIITDY

SEQ ID NO: 33 - protein, 11 aa, Artificial
  synthetic consensus sequence
MPDMTGYDLLK

SEQ ID NO: 34 - protein, 18 aa, Artificial
  synthetic consensus sequence
SSSLKDIPVVIMSSENVP

SEQ ID NO: 35 - protein, 16 aa, Artificial
  synthetic consensus sequence
EGAEDFLLKPVRLADV

SEQ ID NO: 36 - protein, 5 aa, Artificial
  Synthetic consensus sequence
SHVLR

SEQ ID NO: 37 - protein, 162 aa, Arabidopsis thaliana
MTMEQEIEVLDQFPVGMRVLAVDDDQTCLRILQTLLQRCQYHVTTTNQAQTALELLRKNKFDLVISDVDMPDMDGFKLLELVGLEMDLPVIMLSAHSDPKYVMKGVKHGACDYLLKPVRIEELIWQHVVRKSKLKKNKSNVSNGSGNCDKANRKRKEQYEEE

SEQ ID NO: 38 - protein, 182 aa, Arabidopsis thaliana
MAEVMLPRKMEILNHSSKFGSPDPLHVLAVDDSHVDRKFIERLLRVSSCKVTVVDSATRALQYLGVEEKSVGFEDLKVNLIMTDYSMPGMTGYELLKKIKESSAFREVPVVIMSSENILPRIDRCLEEGAEDFLLKPVKLSDVLRDSLMKVEDLSFTKSIQKRELETENVYPVHSQLKRAKI

SEQ ID NO: 39 - protein, 165 aa, Zea mays; identical to SEQ ID NO: 27

SEQ ID NO: 40 - protein, 119 aa, Artificial
  Synthetic Spo0F protein sequence
MMNEKILIVDDQYGIRILLNEVFNKEGYQTFQAAGLQALDIVTRPDLVLLDMKIPGMDGIEILKRMKVIDENIRVIIMTAYGELDMIQESKELGALTHFAKPFDIDEIAVKKYLPLKSN

SEQ ID NO: 41 - protein, 125 aa, Artificial
  Synthetic CheY protein sequence
MADKELKFLVVDDFSTMRRIVRNLLKELGFNNVEEAEDGVDALNKLQGYGFVISDWNMPNMDGLELLKTIRADGAMSALPVLMVTAEAKKENIIAAAQAGASGYVVKPFTAATLKLNKIFEKLGM

SEQ ID NO: 42 - DNA, 217 bp, Zea mays
atggccactcaaagtccccatgttctcgttgtggatatcattccgtgttgattgccttgttgcatcgattgtcctcaatagtttcaacattcgaggtatcatggttatcatgagtttttataataactatcgagtagtttttacgaatgttctaatattatcattttcatattttttataaaaatagagaacattggtacaatattatcttagatat

SEQ ID NO: 43 - DNA, 215 bp, Zea mays; identical to SEQ ID NO: 4
  misc_feature (8)..(8): n is a, c, g, or t

SEQ ID NO: 44 - DNA, 215 bp, Zea mays
  misc_feature (208)..(208): n is a, c, g, or t
atggccactcaaagtccccatgttctcgttgtggatcattcccgtgttgattgccttgttgcatcgattgtcctcaatagtttcaacattcgaggtatcatggttatcatgagtttttataataactatcgagtagtttttacgaatgttgtaatattatcattttcatattttttataaaaatagagaacattggtacaatattatnttagaat

SEQ ID NO: 45 - DNA, 213 bp, Zea mays
  misc_feature (207)..(207): n is a, c, g, or t
atggccactcaaagtccccatgttctcgttgtggatcattcccgtgttgattgccttgttgcatcgattgtcctcaatagtttcaacattcgaggtatcatggttatcatgagtttttataataactatcgagtagtttttacgaatgttctaatattatcattttcatatttttataaaaatagagaatgttggtacaatattatnttagaa

SEQ ID NO: 46 - DNA, 217 bp, Zea diploperennis
  misc_feature (210)..(210): n is a, c, g, or t
atggccactcaaagtccccatggttcttgttgtggatcattcccgtgttgattgccttgttgcatcgattgtcctcaatagtttcaacattcgaggtatcatggttatcatgagtttttataataactatcgagtagtttttatgaatgttctaatattatcattttcatatatttttataaaaatagagaatgttggtacaatattatntcagaat

SEQ ID NO: 47 - protein, 153 aa, Zea mays; identical to SEQ ID NO: 19

SEQ ID NO: 48 - protein, 120 aa, Zea mays
MATQSPHVLVVDHSRVDCLVASIVLNSFNIRVTVAGGAMEALEFLDNSHVDLILTDYCMPDMTGYDLLREVKESPRLKHIPVVITCTDVIPERIIECFEGGAEEYMIKPLKVSDVILSYM

(19) United States
(12) Patent Application Publication
(10) Pub. No.: US 2009/0307795 A1
(43) Pub. Date: Dec. 10, 2009

(54) ZMTCRR-1 PLANT SIGNAL TRANSDUCTION GENE AND PROMOTER

(75) Inventors: Pascual Perez, Chanonat (FR); Wyatt Paul, Aubiere (FR); Luis Miguel Muniz Menendez, Mostoles Madrid (ES); Gregorio Hueros Soto, Meco Madrid (ES)

Correspondence Address: CONNOLLY BOVE LODGE & HUTZ, LLP, P O BOX 2207, WILMINGTON, DE, 19899, US

(73) Assignee: Biogemma, Paris (FR)

(21) Appl. No.: 12/279,273

(22) PCT Filed: Feb. 14, 2007
(86) PCT No.: PCT/EP07/51458

§ 371 Date: Dec. 15, 2008

(30) Foreign Application Priority Data
Feb. 16, 2006 (EP) 06110067.3

(51) Int. Cl. C12N 15/82; C12N 15/11; C12N 15/00; C12N 5/04; A01H 5/00; C07K 14/415

(52) U.S. Cl. 800/278; 536/23.6; 435/320.1; 435/419; 800/298; 800/320.1; 800/320.2; 800/320.3; 800/322; 530/350

(57) ABSTRACT

The present invention relates to the improvement of agronomic qualities of plants. It concerns in particular a nucleic acid molecule encoding a plant signal transduction protein that modulates agronomic qualities of plants.

The present invention relates to the improvement of agronomic qualities of plants. It concerns in particular a nucleic acid molecule encoding a plant signal transduction protein that modulates agronomic qualities of plants.

The endosperm, a characteristic formation of Angiosperm seeds, is a nutritive tissue for the embryo. The maize endosperm originates with series of free-nuclear divisions, followed by cellularisation and the subsequent formation of a range of functional cellular domains. This tissue is complex in its structure and development, in particular in cereals.

The endosperm is the main storage organ in maize seeds, nourishing the embryo while the seed develops, and providing nutrients to the seedling on germination. Thus, the uptake of assimilates by the growing endosperm is a critical process in seed development.

The central area of the endosperm consists of large cells with vacuoles, which store the reserves of starch and proteins, whilst the region surrounding the embryo is distinguished by rather small cells, occupied for the major part by cytoplasm.

The Basal Endosperm Transfer Layer (BETL) area is highly specialized to facilitate uptake of solutes during grain development. These transfer cells of the basal endosperm have specialised internal structures adapted to absorb solutes from the maternal pedicel tissue, and translocate these products to the developing endosperm and embryo.

Transfer cells in maize are a highly specialized tissue in the placental side of the endosperm, forming an interface between the filial and maternal tissues in the seed. They show a differentiated morphology, with deep cell wall ingrowths that allow for a remarkable increase in the membrane surface, thus facilitating nutrient uptake from the apoplastic space at the maternal placento-chalaza (Patrick et al, 2001). These cells also display a specific gene expression program, which has been subject of thorough study in recent years (Hueros et al, 1999b; Gomez et al, 2002), providing evidences for their additional implication in defence of the grain against mother plant-borne pathogens (Serna et al, 2001). Immediately above the transfer cells, a region of prismatic cells known as conductive cells facilitate symplastic transport for the assimilates in their way to the upper part of the endosperm, where they are used to build up storage products.

Response regulators are classical molecules in environmentally controlled processes in bacteria, where they mediate most signal transduction signalling pathways. In plants they are very frequently involved in cytokinin and ethylene-mediated responses (Brandstatter and Kieber., 1998; Sakai et al., 2001).

The improvement of agronomic qualities of plants is still requested by seed companies. Influencing the timing of cellularisation and the extent and duration of endosperm mitosis are ways to improve agronomic qualities of plants, via an improvement of grain yield.

The authors of the present invention have now identified and characterized a response regulator (named ZmTCRR-1) that acts as a signal transduction protein to influence cellularization, mitosis and differentiation of grain tissues.

Such a gene is particularly useful for the improvement of grain yield.

Interestingly, the ZmTCRR-1 nucleotide sequence according to the invention is expressed specifically in the maize kernel transfer cell layer. This is the first tissue-specific response regulator identified to date.

Surprisingly, the authors found that the protein encoded by the ZmTCRR-1 gene, moves from the transfer cells inwards the endosperm tissue, where seems to accumulate in the conducting tissue. This was not previously described for any other response regulator protein or any other protein expressed in the BETL.

It is strongly suggested that ZmTCRR-1 acts in an inter-cellular signalling mechanism, transmitting a differentiation signal initially originated at the border between filial and maternal tissues.

Advantageously, the gene according to the present invention also improves diseases resistance to pathogens.

The present invention relates to an isolated nucleic acid molecule encoding a plant signal transduction protein, comprising a sequence selected from the group consisting of:
a) a nucleotide sequence encoding a protein having the amino acid sequence represented by SEQ ID No: 2;
b) the nucleotide sequence represented by SEQ ID No: 1;
c) a sequence hybridizing under stringent conditions with the complementary strand of a nucleic acid molecule as defined in (a) or (b), and coding for a protein having signal transduction activity.

The present invention also relates to a nucleotide sequence encoding a protein having the amino acid sequence SEQ ID No: 2 wherein the Asp (Aspartate) in position 58 is replaced by Glu (Glutamate) at the same position. This leads to a more effective plant signal transduction protein (constitutively active form).

The invention also relates to the protein having the amino acid sequence SEQ ID No: 2 wherein Asp (Aspartate) in position 58 is replaced by Glu (Glutamate) at the same position.

Brodaly, all the aspects of the invention as described for the “original” TCRR1 protein (SEQ ID N^{o} 2) are inter alia applicable to said modified sequence.

“Homologous nucleic acid sequence”, or “homologous DNA sequence”, means any nucleic acid sequence which differs from the sequence SEQ ID No: 1 by a substitution, deletion and/or insertion of one or more nucleotides at positions such that these homologous nucleic acid sequences preserve the signal transduction property of sequence SEQ ID No: 1.

Preferably such a homologous nucleic acid sequence is at least 70% identical to the sequence SEQ ID No: 1, preferably at least 85% identical, more preferably at least 90, 91, 95, 98, 99.9% identical. Also preferably, the degree of identity is defined by comparison with the entire sequence of reference, SEQ ID No: 1.

Homology is generally determined using a sequence analysis software (for example, the Sequence Analysis Software package of the Genetics Computer Group, University of Wisconsin Biotechnology Center, 1710 University Avenue, Madison, Wis. 53705). Similar nucleotide sequences are aligned in order to obtain the maximum degree of homology (i.e. identity). To this end, it may be necessary to artificially introduce gaps in the sequence. Once the optimum alignment has been achieved, the degree of homology (i.e. identity) is established by recording all the positions for which the nucleotides of the two compared sequences are identical, with respect to the total number of positions.

In a preferential manner such a homologous nucleic acid sequence specifically hybridizes to a sequence which is complementary to the sequence SEQ ID No: 1 under stringent conditions. The parameters defining the stringency conditions depend on the temperature at which 50% of the paired strands separate (Tm).

For sequences comprising more than 30 bases, Tm is defined by the equation: Tm=81.5+0.41 (% G+C)+16.6 Log(concentration in cations)−0.63 (% formamide)−(600/number of bases) (Sambrook et al., 1989).

For sequences shorter than 30 bases, Tm is defined by the equation: Tm=4(G+C)+2(A+T).

Under appropriate stringency conditions, in which non-specific (aspecific) sequences do not hybridize, the temperature of hybridization is approximately between 5 and 30° C., preferably between 5 and 10° C. below Tm and hybridization buffers used are preferably solutions of higher ionic force like a solution 6*SSC for example.

Preferably, the nucleic acid molecule encoding the plant signal transduction protein according to the invention (ZmTCRR-1) consists in the nucleotide sequence represented by SEQ ID No: 1.

The nucleic acid molecule encoding a plant signal transduction protein according to the invention can be isolated from various plant species, notably Angiosperm plants, Monocotyledons as Dicotyledons and are preferably nucleic acid molecules isolated from a plant selected from the group consisting of maize, teosintes, wheat, barley, rye, rice, sorghum, and sugar cane. Preferably said plant is maize.

The present invention also relates to maize allelic variants of SEQ ID No: 1.

Two maize genes are allelic variants if they both come from the same species (maize), have the same function (plant signal transduction protein according to this invention), are usually localized at the same region in the same chromosome, (although chromosomal translocations may occur), but originate from different lines, and differs between them by the presence of indels (deletions, insertions) or SNPs (Single Nucleotide Polymorphisms). Allelic variants of ZmTCRR-1 (SEQ ID No: 1) could be obtained easily by the man skilled in the art using PCR, genomic hybridization, and representative examples of that genus are given in FIG. 2 (partial sequences).

Another object of the present invention is a nucleotide construction, referred to as an expression cassette, comprising a nucleic acid molecule encoding a plant signal transduction protein as defined above, operatively linked to regulatory elements allowing the expression in prokaryotic and/or eukaryotic host cells. Regulatory elements allowing expression of genes are notably 5′ and 3′ regulatory sequences.

“Operatively linked” refers to functional linkage between the 5′ and 3′ regulatory sequences and the controlled nucleic acid sequence according to the invention.

The 5′ regulatory sequences are notably promoters.

Any suitable promoter could be used. It could be a constitutive promoter. It could also be for example a tissue-specific promoter such as a root-specific promoter, a leaf-specific promoter, a seed-specific, a BETL specific etc. Numerous tissue-specific promoters are described in the literature and any one of them can be used.

Examples of promoters useful for plant transformation include the 35S promoter or the 19S promoter (Kay et al., 1987), the pCRV promoter (Depigny-This et al., 1992), the ubiquitin 1 promoter of maize (Christensen et al., 1996), the regulatory sequences of the T-DNA of Agrobacterium tumefaciens, including mannopine synthase, nopaline synthase, octopine synthase, the promoters regulated during seed development such as the HMWG promoter (High Molecular Weight Glutenin) of wheat (Anderson O. D. et al., 1989, Roberts et al., 1989), the waxy, zein or bronze promoters of maize, a promoter that is inducible by pathogens.

Preferably, the promoter is a pathogen inducible promoter. Such promoters include those from pathogenesis-related protein, which are induced following infection by a pathogen, e.g., PR proteins, SAR proteins, beta-1,3 glucanase, chitinase, etc.

Still preferably, the promoter is a BETL-specific promoter such as BETL-1 (Hueros et al, 1995, 1999b) or BETL-2 (WO 99/50427) promoter.

When a BETL-specific promoter is used, it is also in the scope of the present invention to co-transform the plant with both the expression cassette comprising the ZmTCRR-1 gene under control of the BETL-specific promoter and an expression cassette comprising the ZmMRP1 factor. Accordingly ZmMRP1 will transactivate the BETL-specific promote leading to an increase expression of the ZmTCRR-1 gene.

The 3′ regulatory sequences are notably terminators.

Among the terminators useful for plant transformation within the framework of the present invention, the ones which can be used are the polyA 35S terminator of the cauliflower mosaic virus (CaMV), described in the article of Franck et al. (1980), the NOS terminator corresponding to the region in the non coding 3′ region of the nopaline synthase gene of the Ti-plasmid of Agrobacterium tumefaciens nopaline strain (Depicker et al. 1992), the histone terminator (EP 0 633 317), and the tml terminator.

Preferentially the terminator is the 3′Nos or 3′CaMV terminator.

Any other element like introns, enhancers, transit peptides, etc. . . . may be comprised in the expression cassette. Introns and enhancers may be used to improve the expression of the gene according to the present invention.

Among useful introns, the first intron of maize adh1S can be placed between the promoter and the coding sequence. This intron when included in a gene construct increased the expression of the desired protein in maize cells (Callis et al., 1987). One also can use the 1^{st} intron of the shrunken 1 gene of the maize (Maas et al., 1991), the 1^{st} intron of the catalase gene of the bean catalase (CAT-1) (Ohta et al., 1990), the 2^{nd} intron of the ST-LS1 gene of potato (Vancanneyt et al. 1990), the DSV intron of the yellow dwarf virus of tobacco (Morris et al., 1992), the actin-1 intron (act-1) of rice (McElroy et al., 1990) and intron 1 of triosephosphate isomerase (TPI) (Snowdon et al., 1996). Preferentially, the intron used in the present invention is the Hsp70 intron or the Sh1 intron.

The expression cassettes may additionally contain 5′ leader sequences. Such leader sequences can act to enhance translation. Such 5′ leaders are known in the art and include, but are not limited to, picornavirus leaders, for example, the EMCV leader (Encephalomyocarditis 5′ noncoding region) (Elroy-Stein, Fuerest, and Moss B., 1989); potyvirus leaders, for example, the TEV leader (Tobacco etch Virus) (Allison et al., 1986); the human immunoglobulin heavy-chain binding protein leader (BiP) (Macejack and Sarnow, 1991); the untranslated leader from the coat protein mRNA of alfalfa mosaic virus (AMV RNA 4) (Jobling and Gehrke, 1987); the tobacco mosaic virus leader (TMV) (Gallie et al., 1989); and the maize chlorotic mottle virus leader (MCMV) (Lommel et al., 1991). See also, Della-Cioppa et al. (1987). Other methods known to enhance translation can be utilized, for example introns, and the like.

According to the invention, the expression cassette, comprising a nucleic acid molecule encoding a plant signal transduction protein as defined above, operatively linked to regulatory elements allowing the expression in prokaryotic and/or eukaryotic host cells may further comprises one or several selection marker gene for plants, useful for transformation and selection.

In the present invention, the term “selectable marker”, “selectable gene”, “selectable marker gene”, “selection marker gene”, “marker gene” are used interchangeably.

These selectable markers include, but are not limited to, antibiotic resistance genes, herbicide resistance genes or visible marker genes. Other phenotypic markers are known in the art and may be used in this invention.

A number of selective agents and resistance genes are known in the art. (See, for example, Hauptmann et al., 1988; Dekeyser et al., 1988; Eichholtz et al., 1987 ; and Meijer et al., 1991).

Notably the selectable marker used can be the bar gene conferring resistance to bialaphos (White et al., 1990), the sulfonamide herbicide Asulam resistance gene, sul (described in WO 98/49316) encoding a type I dihydropterate synthase (DHPS), the nptll gene conferring resistance to a group of antibiotics including kanamycin, G418, paromomycin and neomycin (Bevan et al., 1983), the hph gene conferring resistance to hygromycin (Gritz et al., 1983), the EPSPS gene conferring tolerance to glyphosate (U.S. Pat. No. 5,188,642), the HPPD gene conferring resistance to isoxazoles (WO 96/38567), the gene encoding for the GUS enzyme, the green fluorescent protein (GFP), expression of which, confers a recognisible physical characteristic to transformed cells, the chloramphenicol transferase gene, expression of which, detoxifies chloramphenicol.

Advantageously, the selectable marker gene is inserted between a promoter and a terminator in a second expression cassette. Said second expression cassette being integrated in the same vector as the expression cassette containing the gene according to the invention.

According to this advantageous embodiment, the marker gene is preferably controlled by a promoter which allows expression in cells, thus allowing selection of cells or tissue containing the marker at any stage of development of the plant. Preferred promoters are the promoter of nopaline synthase gene of Agrobacterium, the promoter derived from the gene which encodes the 35S subunit of cauliflower mosaic virus (CaMV) coat protein, and the rice actin promoter. However, any other suitable second promoter may be used. Any terminator may be used. Preferred terminators are the 3′CaMV and Nos terminator as previously described.

Advantageously, the expression cassette containing the selectable marker gene is comprised between two Ds elements (transposons) in order for its removal at a later stage by interacting with the Ac transposase. This elimination system is described in Yoder et al. (1993).

In preparing the expression cassettes, the various DNA sequences or fragments may be manipulated, so as to provide DNA sequences or fragments in the proper orientation and, as appropriate, in the proper reading frame. Towards this end, adapters or linkers may be employed to join the DNA fragments and/or other manipulations may be required to provide convenient restriction sites, removal of superfluous DNA, removal of restriction sites, or the like. For this purpose, in vitro mutagenesis, primer repair, restriction, annealing, ligation, PCR, or the like may be employed, where nucleotide insertions, deletions or substitutions, for example transitions and transversions, may be involved. These techniques are well known by those skilled in the art.

Another object of the invention is any nucleotide vector referred to as an expression vector, such as a plasmid, which can be used for transforming host cells, characterized in that it contains at least an expression cassette (as described above) comprising a nucleic acid molecule encoding a signal transduction protein, as defined above.

The construction of expression vectors for the transformation is within the capability of one skilled in the art following standard techniques.

The decision as to whether to use a vector, or which vector to use, is guided by the method of transformation selected, and by the host cell selected.

Where a naked nucleic acid introduction method is used, then the vector can be the minimal nucleic acid sequences necessary to confer the desired phenotype, without the need for additional sequences.

Possible vectors include the Ti plasmid vectors, shuttle vectors designed merely to maximally yield high numbers of copies, episomal vectors containing minimal sequences necessary for ultimate replication once transformation has occured, transposon vectors, including the possibility of RNA forms of the gene sequences. The selection of vectors and methods to construct them are commonly known to persons of ordinary skill in the art and are described in general technical references (Mullis, K B (1987), Methods in Enzymology).

For other transformation methods requiring a vector, selection of an appropriate vector is relatively simple, as the constraints are minimal. The apparent minimal traits of the vector are that the desired nucleic acid sequence be introduced in a relatively intact state. Thus, any vector which produces a plant carrying the introduced DNA sequence should be sufficient. Also, any vector which introduces a substantially intact RNA which can ultimately be converted into a stably maintained DNA sequence should be acceptable.

However, any additional attached vector sequences which confer resistance to degradation of the nucleic acid fragment to be introduced, which assists in the process of genomic integration or provides a means to easily select for those cells or plants which are actually, in fact, transformed are advantageous and greatly decrease the difficulty of selecting useable transgenic plants.

The vector can exist, for example, in the form of a phage, a plasmid or a cosmid. The construction of such expression vectors for transformation is well known in the art and uses standard techniques. Mention may be made of the methods described by Sambrook et al. (1989).

Another object of the invention is a host cell, containing at least an expression vector as described above.

The decision as to whether to use a host cell, or which host cell to use, is guided by the method of transformation.

The host cell can be any prokaryotic or eukaryotic cell. Any of a large number of available and well-known host cells may be used in the practice of this invention. The selection of a particular host is dependent upon a number of factors recognized by the art. These include, for example, compatibility with the chosen expression vector, bio-safety and costs. Useful hosts include bacteria such as E. coli sp. or Agrobacterium. A plant host cell, may be also used, notably an Angiosperm plant cell, Monocotyledon as Dicotyledon plant cell, particularly a cereal or oily plant cell, selected in particular from the group consisting of maize, wheat, barley, rice, rape and sunflower, preferentially maize.

More particularly, the host cell used in carrying out the invention is Agrobacterium tumefaciens, according to the method described in the article of An et al., 1986, or Agrobacterium rhizogenes, according to the method described in the article of Jouanin et al., 1987.

The invention also relates to a transgenic plant, or a part of a transgenic plant (leaves, plant cell, plant tissue, grain, fruit, seed, . . . ) comprising a cell as described, notably comprising stably integrated into the genome a nucleic acid molecule encoding a plant signal transduction protein as identified above, operatively linked to regulatory elements allowing transcription and/or expression of the nucleic acid molecule in plant cells.

A plant or part of a plant (plant cell, plant tissue, grain, seed, fruit, leaves, . . . ) according to the invention could be a plant or a part of it from various species, notably an Angiosperm, Monocotyledons or Dicotyledons, preferably a cereal or oily plant, selected in particular from the group consisting of maize, rice, wheat, barley, rape, and sunflower, preferentially maize.

As used herein, the term “oily plant” denotes a plant that is capable of producing oil, and preferably that is cultivated for oil production.

When a plant already comprises in its genome an endogenous copy of the ZmTCRR-1 gene, the transgenic plant will contain at least one supplemental copy of said gene.

In yet another aspect, the invention also relates to harvestable parts and to propagation material of the transgenic plants according to the invention which either contain transgenic plant cells expressing a nucleic acid molecule according to the invention or which contain cells which show a reduced level of the described protein. Harvestable parts can be in principle any useful parts of a plant, for example, leaves, stems, fruit, seeds, roots etc. Propagation material inclues, for example, seeds, fruits, cuttings, seedlings, tubers, rootstocks etc.

The invention further relates to a plant signal transduction protein or an immunologically or biologically active fragment thereof encodable by a nucleic acid molecule according to the invention.

The invention also relates to an antibody specifically recognizing a plant signal transduction protein according to the invention or a fragment, or epitope thereof.

These antibodies can be monoclonal antibodies, polyclonal antibodies or synthetic antibodies as well as fragments of antibodies, such as Fab, Fv or scFv fragments etc. Techniques for producing such antibodies are classical methods well known by the one skilled in the art.

An other object of the invention is a method of obtaining a plant having improved agronomic qualities, comprising the steps consisting of:
a) transforming at least a plant cell by means of at least a vector as defined previously;
b) cultivating the cell(s) thus transformed so as to generate a plant containing in its genome at least an expression cassette according to the invention, whereby said plant has improved agronomic qualities.

According to the invention, “improved agronomic qualities” means improved agronomic qualities and/or improved nutritional qualities, notably yield, food or industrial qualities of a plant or a part thereof, in comparison with a non-transformed plant that do not contain the heterologous expression cassette of the invention.

Yield could be improved notably by increasing grain size, grain weight, grain mass, and/or improving grain filling, as compared with wild type plants.

So a method for increasing plant grain size, a method for increasing plant grain weight and a method for improving plant grain filling are also in the scope of the present invention.

The agronomic quality of a plant is improved by acting in particular on the size of the embryo or of the endosperm and/or its development. A gene according to the invention will influence the process of endosperm cellularisation, cell division and differentiation and thus the development of the endosperm. As a consequence there is an effect on the accumulation of nutrients in the embryo and endosperm.

The transformation of vegetable cells can be achieved by any one of the techniques known to one skilled in the art.

It is possible to cite in particular the methods of direct transfer of genes such as direct micro-injection into plant embryoids (Neuhaus et coll. 1997), vacuum infiltration (Bechtold at al. 1993) or electroporation (Chupeau et coll., 1989) or direct precipitation by means of PEG (Schocher et coll., 1986) or the bombardment by gun of particules covered with the plasmidic DNA of interest (Fromm M et al., 1990).

It is also possible to infect the plant with a bacterial strain, in particular Agrobacterium. According to one embodiment of the method of the invention, the vegetable cells are transformed by a vector according to the invention, the said cell host being able to infect the said vegetable cells by allowing the integration, in the genome of the latter, of the nucleotide sequences of interest initially contained in the above-mentioned vector genome. Advantageously, the above-mentioned cell host used is Agrobacterium tumefaciens, in particular according to the method described in the article by An et al., (1986), or Agrobacterium rhizogene, in particular according to the method described in the article by Guerche et al. (1987).

For example, the transformation of vegetable cells can be achieved by the transfer of the T region of the tumour-inducing extra-chromosome circular plasmid of Agrobacterium tumefaciens, using a binary system (Watson et al., 1994). To do this, two vectors are constructed. In one of these vectors the T region has been eliminated by deletion, with exception of the right and left borders, a marker gene being inserted between them to allow selection in the plant cells. The other partner of the binary system is an auxiliary plasmid Ti, a modified plasmid which no longer has any T region but still contains the virulence genes vir necessary to the transformation of the vegetable cell.

According to a preferred mode, it is possible to use the method described by Ishida et al. (1996) for the transformation of Monocotyledons.

According to another protocol, the transformation is achieved according to the method described by Finer et al. (1992) using the tungsten or gold particle gun.

Selection:

The engineered plant material may be selected or screened for transformants (those that have incorporated or integrated the introduced nucleotide construction(s)). Such selection and screening methodologies are well known to those skilled in the art. The selection and screening method is chosen depending on the marker gene used.

An isolated transformant may then be regenerated into a plant.

Regeneration:

Normally, regeneration is involved in obtaining a whole plant from the transformation process. The term “regeneration” as used herein, means growing a whole plant cell, a group of plant cells, a plant part or a plant piece (for example, from a protoplast, callus, or tissue part).

Methods of regenerating whole plants from plant cells are known in the art, and the method of obtaining transformed and regenerated plants is not critical to this invention.

In general, transformed plant cells are cultured in an appropriate medium, which may contain selective agents such as antibiotics, where selectable markers are used to facilitate identification, of transformed plant cells. Once callus forms, shoot formation can be encouraged by employing appropriate plant hormones in accordance with known methods and shoots transferred to rooting medium for regeneration of plants. The plants may then be used to establish repetitive generations, either from seeds or using vegetative propagation techniques.

The invention further relates to the use of at least an expression cassette as previously defined, for obtaining a transgenic plant exhibiting improved agronomic qualities.

The present invention can also be used in the context of the selection of plants having improved yield and agronomic qualities.

They are of most particular value in the context of marker-assisted selection (MAS), which makes it possible to use accelerated backcross techniques consisting in using the linkage which exists between a molecular marker and a allele of agronomic interest, in this case encoding ZmTCRR1, for transferring said allele of interest into various genotypes in order to provide them with increased yield and improved agronomic qualities.

The present invention can also be used to monitor the integration of an allele of a ZmTCRR1 gene that is favourable to improved yield and increased agronomic qualities (as compared with the same plant that do not contain the favourable allele), for example in the context of introgression techniques by means of successive crosses between plants having this allele.

The invention also relates to seeds obtained from a plant transformed with a nucleic acid sequence according to the invention (SEQ ID No: 1).

The products obtained, whether it be seeds with higher oil content, flours of seeds or grains with a higher starch, protein or oil content, bigger size, bigger weight (as compared with a non transformed plant), also come within the scope of the invention.

The invention also provides any composition for human or animal food prepared from the said obtained products.

The present invention also relates to the ZmTCRR-1 promoter sequence represented by SEQ ID No: 8.

This promoter is useful for driving expression of genes in the BETL. Constructs comprising such a promoter and a gene of interest (linked to improved yield, disease resistance) and transformed plants comprising said genetic construct are also part of the invention.

The gene of interest can be of a heterologous origin, and can be placed in the sense or antisense orientation.

According to an embodiment, the gene of interest may be selected from the group consisting of a sequence that encode a peptide or a protein, an antisense RNA sequence, a sense RNA sequence, both a sense and antisense RNA sequence and/or a ribozyme.

Preferentially, the gene of interest is a sequence that codes for a protein or for a peptide.

The said gene of interest can for example code for a protein involved in the development of the embryo and/or of the endosperm, the determination of seed size and/or quality (e.g. MRP1 or Ferretin (Lobreaux S. et al. 1992)), cell growth (proteins regulating cell division including cytokinin or auxin genes, e.g. ipt (Zhang et al. 1995), the flow of nutrients or nutrient transfer (transporters (Bolchi A. et al. 1999)), proteins involved in fatty acids metabolism. The gene of interest may also encode an enzyme involved in sugar metabolism such as invertases (e.g. incW2 (Taliercio EW et al. 1999)), sucrose synthases (e.g. Sh1), the saccharose phosphate synthase, saccharose synthase, UDP-Glucose pyrophosphorylase, ADP-glucose pyrophosphorylase (Thomas W. Greene et al. 1998), starch branching enzyme (Ming Gao et al. 1997) or the starch synthase (Mary E. Knight et al. 1998). The gene of interest could also code for a hexokinase as the one described by Jang et al. (1997) in order to improve grain filling. The gene of interest may additionally code for a protein that is involved in amino acids transfer, such as a methionine permease or a lysine permease, or a sulphur transporter etc. It can also code for a toxic protein such as Barnase, for a protein activating or inhibiting other genes, such as transcriptional regulators including transactivators modified to act as dominant activators or repressors of transcription (e.g. fusions to the engrailed domain (Poole et al., 1985) or co-repressors for example), or for a protein improving resistance to pathogens (e.g. BAP2, MRP1).

Preferably, said gene of interest encodes a protein selected from:

a protein whose specific expression in the endosperm, and particularly in the BETL, makes it possible to increase nutrient uptake and thus seed size and/or quality; examples of such a protein include an invertase like Incw2 or like Ivr1 (EP 0 442 592), a sucrose synthase like Sh1 (WO 02/067662) or any transporters of sugar and nitrogen or a MRP1 protein etc;

a protein that improves resistance to pathogens; examples of such a protein include a BAP Protein (Basal Layer Antifungal Protein) (Serna et al., 2001), or anti-fungal peptides, or a MRP1 protein or a protein that encodes an oxalate oxidase (WO 92/15685) or a protein that encodes a chitinase (WO 92/01792 or U.S. Pat. No. 5,446,138) or a protein that encodes a glucanase (WO 93/02197) etc.

A protein that “improves resistance to pathogens” or “a protein improving resistance to pathogens” means a protein that, when expressed in a plant or a part of a plant, confers or improves resistance to pathogens to said plant, or part thereof. Said transformed plant has a better resistance to pathogens than the non-transformed plant (wild-type).

The said gene of interest can also be associated with other regulating elements such as transcription termination sequences (terminators). By way of examples of such sequences, it is possible to cite the polyA 35S terminator of the cauliflower mosaic virus (CaMV), described in the article of Franck et al. (1980) and the NOS terminator corresponding to the region in the non-coding 3′ region of the nopaline synthase gene of the Ti-plasmid of the Agrobacterium tumefaciens nopaline strain (Depicker et al. 1992).

Preferably, the terminator used is the 3′CaMV.

The present invention will be further understood in view of the annexed figures and following examples.

### FIGURES LEGENDS

FIG. 1—a) Alignment of the amino acid sequences of maize response regulator proteins. Only the conserved N-terminal regions are shown. b) Dendrogram of alignments (entire proteins).

FIG. 2—Alignment of ZmTCRR-1 to Response Regulators or response regulator domains in plant and bacteria. Fully conserved residues are marked in black. Asterisks denote aminoacids forming the acidic pocket. A variation in the canonical D13 (measured in the CheY sequence) is underlined in the ZmTCRR-1 sequence. Only domains relevant for the alignement are presented for ARR10, ARR6 and ZmRR8. Acession numbers: ARR10, O49397; ARR6, NP_{—}201097; ZmRR1, BAA85112; ZmRR8, BAB41137.1; Spo0F, P06628; CheY, P96126.

FIG. 3—Alignment of the DNA sequences of the 5′ coding region of 4 maize inbred lines and the wild relative of maize teosinte. The Amino acid Histidine at position 13 is conserved.

FIG. 4—ZmTCRR-1 is a single copy gene in maize. 15 micrograms of maize genomic DNA were digested with four different endonucleases, transferred to nylon membranes and hybridized with a DIG labelled probe 38-10. A single, distinct band was observed in each lane. B: BamHI; E-I: EcoR-I; E-V: EcoR-V; H: HindIII.

FIG. 5—Schematic of ZmTCRR-1 gene organisation.

FIG. 6—Expression pattern of ZmTCRR-1 in different tissues and kernel development stages. Total RNA from different corn tissues (A) and seed developmental stages (B) were hybridized with the probe 38-10. U: unpollinated flowers; T: top half of the seed; B: Bottom half of the seed; L: leaves; R: roots; C: coleoptiles; A: anthers; S: silks. 3-32 DAP: days after pollination.

FIG. 7—Western blot analyses of the localisation of ZmTCRR-1. Total protein extracts from 8, 11 or 16 DAP upper (T) or lower (B) halves were reacted with the Anti-ZmTCRR1 antibody (RR), a pre-immune serum (Pre) or the antibodies against the transfer cell specific proteins BETL-1 (B1) and BAP-2 (B2). Rec, 100 ng of the recombinant ZmTCRR-1 that was used to raise the antibody. MWM, a lane containing molecular weight markers. Only the blot area between 3 and 10 KDa is shown. Arrows in panel B2 indicate the low molecular weight product that corresponds to the mature BAP-2 protein.

FIG. 8—Subcellular location of ZmTCRR-1. ZmTCRR-1-GFP translational fussions were transformed into onion epithelial cells (A to C) and tobacco protoplasts (D, E) via helium bombardment and chemical transformation, respectively. C and E, GFP controls: A, B, and D, ZmTCRR-1-GFP. D includes 3 images of the same protoplast, focused in different planes.

FIG. 9—Transactivation of ZmTCRR-1 promoter by ZmMRP-1. Panel 1. The ZmTCRR-1 promoter was fused to the GUS gene and co-bombarded into onion epithelia, together with a plasmid bearing (B) or not (A) the ZmMRP1 coding sequence under the control of the ubiquitin promoter. An ubiquitin promoter-GUS construct (C) was introduced as a positive control. An area of 1-2 squared cm, having the highest density of blue spots found in the sample, is shown in each case. Panel 2. The ZmTCRR1prom-GUS construct was cotransformed into tobacco protoplasts, together with a plasmid bearing (+) or not (−) the ZmMRP1 coding sequence under the control of the ubiquitin promoter, a 35S-LUC construct was also introduced as an internal control. Ratios GUS/LUC (stripped bars) are the average of 5 independent assays. A BETL-1 promoter-GUS construct was also assayed (solid bars).

FIG. 10—Schematic diagrams of binary vector constructs used for maize plant transformation A) Constitutive ZmTCRR-1 expression vector B) BETL-specific ZmTCRR-1 expression vector.

FIG. 11—Schematic diagrams of binary vector constructs used for maize plant transformation A) Constitutive ZmTCRR-1 RNAi expression vector B) BETL-specific ZmTCRR-1 RNAi expression vector with pBETL1 C) BETL-specific ZmTCRR-1 RNAi expression vector with pBETL9.

FIG. 12—Comparison of transgenic (T) and non-transgenic (WT) maize seed A) weight and B) size. The transgenic maize seeds comprise and express the ZmTCRR-1 gene under the control of the maize BETL9 promoter.

Boxplot interpretation: The box itself represents the middle 50% of the data. The triangle in the box indicates the median value of the data. The ends of the vertical lines indicate the minimum and maximum data values.

### EXAMPLES

The invention will now be described by the way of the following examples, which should not be construed as in any way limiting the scope of the invention.

### Plant Material:

DNA and RNA samples were obtained from greenhouse grown maize plants (inbred lines A69Y, B73, F2, W64 and the wild maize ancestor teosinte). Tobacco protoplasts were obtained from the “Petit Havana” variety, grown under greenhouse conditions.

### Example 1

### Identification and Characterization of the ZmTCRR-1 Gene and Protein (Zea mays Transfer Cell Response Regulator-1)

1.1. ZmTCRR-1 Gene Sequence Identification:

An expression database has been built for over 6000 transcripts randomly selected from a 10 days after pollination (10 DAP) endosperm cDNA library, using a differential screening approach. The membranes were hybridised with subtracted probes enriched for transcripts specific for different domains and developmental stages of the kernel.

cDNA Library Preparation and Subtracted Probe Preparation:

A lambda 10 DAP (days after pollination) kernel library from inbred line A69Y (described in Hueros et al., 1995) was converted to plasmid and amplified in DH10B cells. Plasmids were digested with Notl to obtain linear fragments, which were size-fractionated in 1% agarose. Linear plasmids containing inserts between 0.5-1.0 kb and 1.0-2.0 kb were excised from the gel in two pools, religated and transformed into DH10B cells by electroporation. Transformants were then plated on LB-ampicillin plates for colony isolation.

RNA obtained from 8 DAP seeds, 21 DAP seeds and top or bottom half of 10 DAP seeds was used to synthesize subtracted probes for each of these conditions using the PCR-Select kit (Clontech).

### Differential Display:

6000 clones from the above mentioned library were randomly picked and used for PCR amplification of their insert with universal/reverse primers for pBluescript. The reactions were electrophoresed in 1.5% agarose and transferred to charged nylon membranes (Roche). These filters were hybridised with ³²P-labelled probes obtained by random primer (RediPrime II Labelling kit, Amersham) from the 8 DAP, 21 DAP, top and bottom subtracted cDNA samples and a mixed roots+leaves unsubstracted cDNA sample in order to identify transcripts with preferential expression in a defined tissue/time frame. The hybridisation signal from each clone to each sample was subjectively recorded in a range from no signal (−) to very strong signal (++++).

Clone 3810 showed moderate hybridisation to the subtracted basal kernel specific probe and no signal with any other probe in the set. This clone carries a 410 bp long insert. The insert in clone 3810 included the 45 C-terminal residues of the protein and a 230 bp 3′UTR.

Isolation of a Full cDNA Corresponding to Clone 3810:

To obtain the 5′ terminus of the cDNA the Advantage2 cDNA RACE PCR kit (Clontech) was used, following the manufacturer's instructions. Once the 5′ end was obtained, new primers were designed and the full cDNA was amplified from 10 DAP seed mRNA. The primers used were RRfw (5′ CTAGTCCATGGCCACTCAAAGTCC 3′, SEQ ID N^{o} 10) and 3810rw (5′ AGGCTTGCATTGGCTACAAATTATTC 3′, SEQ ID N^{o} 11). The putative full length cDNA obtained was 626 bp long, encoding a 124 aa peptide with a predicted molecular weight of 13.8 kDa and predicted pl of 4.79 (SEQ ID N^{o} 1)

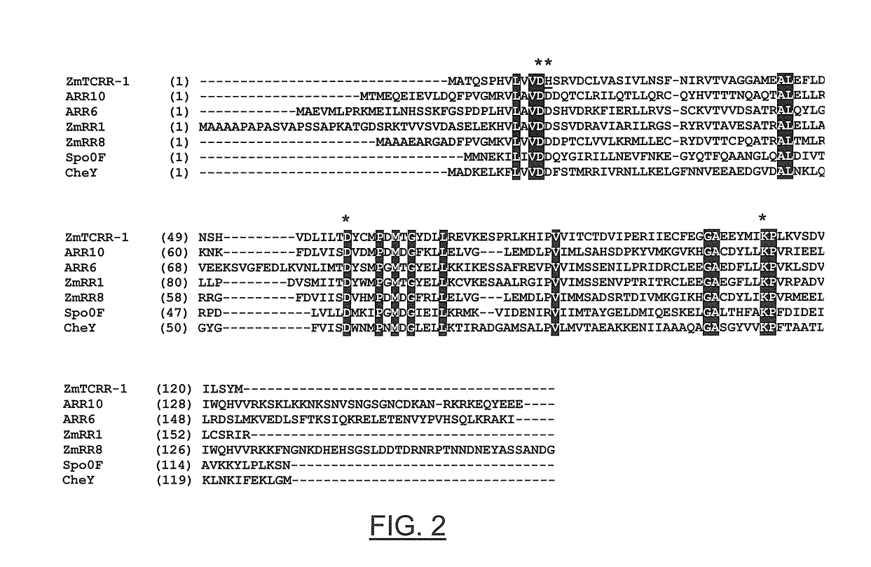

A motif search using PFAM indicates the presence of a response regulator domain in the region 6-119 of the predicted protein (E(0)=5e-8). A pair-wise sequence comparison of ZmRRs 1-10, all of them type-A response regulator molecules (Asakura et al., 2003), to ZmTCRR-1 produces identities between 21.1% and 42.6% (FIG. 2). When the whole group is considered, identity descends to 12.5%, except in regions highly conserved in type-A response regulators (Hwang et al., 2002). Thus ZmTCRR-1 represents a new member of the maize RR family. Almost all the protein is included in the response regulator domain, with a very short C-terminal extension. This strongly suggests that the motif itself is responsible for the protein's function, instead of regulating an adjacent domain as is usually the case in other type-A RRs in plants, with C-terminal domains ranging from 30 to 100 aminoacids (D'Agostino and Kieber, 1999).

ZmTCRR-1 shows the conserved residues archetypical of type-A RRs with one exception: Aspartic 13 is substituted by a histidine. An alignment of its sequence to response regulators from plants and bacteria present in Swissprot and NCBI databases shows a high sequence conservation in the regions around the aa involved in the phosphate group transfer activity (acidic pocket, FIG. 2). However, one of the canonical aspartic residues in this structure, D13, is changed to histidine in ZmTCRR-1. This variation of the canonical motif allows us to separate ZmTCRR-1 from the other known plant response regulators, which show the classical DDK triad.

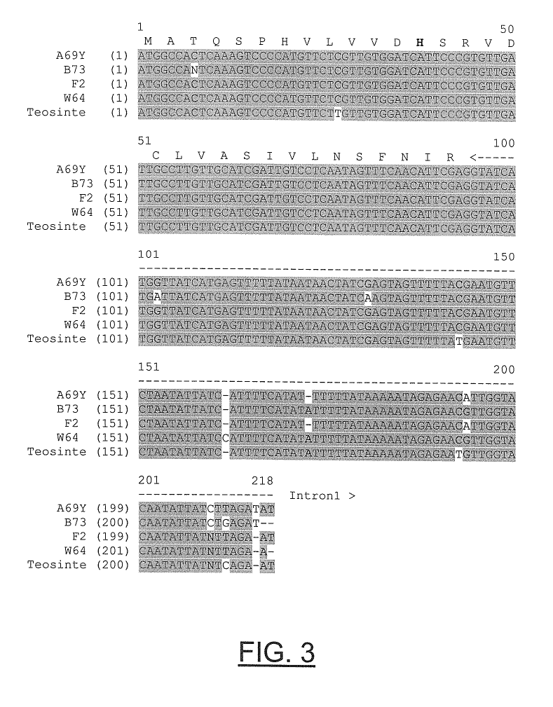

A region from the start codon to base 220 inside the first intron was sequenced in the inbred lines B73, F2, W64 and the maize ancestor teosinte (Zea diploperennis). The alignment of the sequences showed that the transversion was present in all the samples, which were 97.9% identical in the exonic region, with full conservation of the aminoacid sequence (FIG. 3).

The sequence was scanned for transmembrane domains using Tmphred (Hofmann and Stoffel, 1993), SOSUI (http://sosui.proteome.bio.tuat.ac.jp) and signal peptides using TargetP v1.0 (Emanuelsson et al., 2000) and Predotar v0.5 (http://www.inra.fr/predotar) programs. No targeting sequence to any cell compartment was found.

### 1.2. ZmTCRR-1 is a Single Copy Gene in Maize:

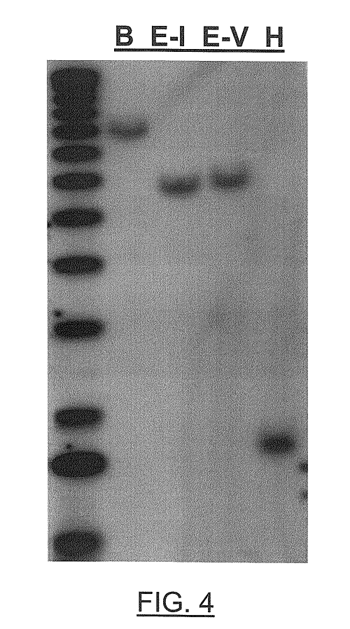

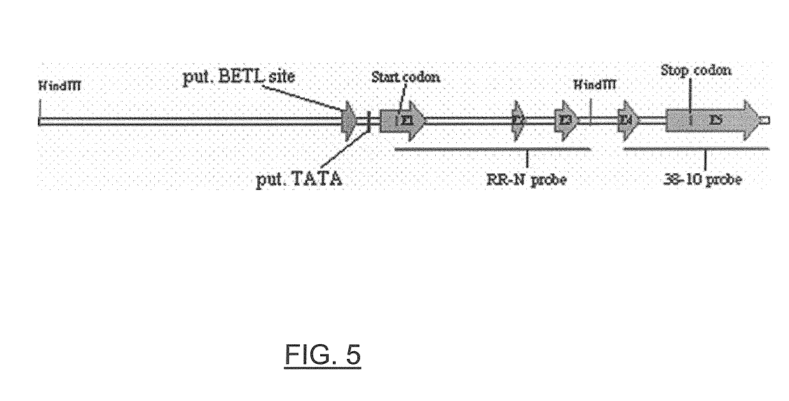

The insert in the clone 3810 was dig-labelled and used to determine the copy number by hybridisation to A69Y genomic DNA digested with different restriction endonucleases. 20 micrograms of DNA were digested with BamHI, HindIII, EcoRI and EcoRV endonucleases, electrophoresed and transferred to charged nylon membranes (Roche). The probe hybridised to a single DNA fragment in all cases, indicating that ZmTCRR-1 is a single copy gene in maize (FIG. 4).

### 1.3. Isolation of the Genomic Sequence of ZmTCRR-1

The primers RRfw (5′ CTAGTCCATGGCCACTCAAAGTCC 3′) and 3810rw (5′ AGGCTTGCATTGGCTACAAATTATTC 3′) already mentioned, encompassing the full CDS and part of the 3′UTR of 3810RR were used to isolate the genomic sequence of the gene by PCR from inbred line A69Y. The amplified fragment of approximately 1200 bp was ligated into the EcoRV site of pBluescript, sequenced and aligned to the cDNA and four introns were found with canonical GT/AG borders. To obtain the promoter sequence of ZmTCRR-1 an inverse PCR strategy was followed. Southern-blot analyses using several restriction enzymes lead to the identification of HindIII as the enzyme producing the most suitable fragment for promoter isolation. Genomic DNA was digested with HindIII and fragments between 1.6 and 2.0 kb were gel-purified, self-ligated and used as template for a PCR reaction using a proofreading enzyme (KOD Hot-Start DNA Polymerase, Novagen). The following nested primers were used in amplifications.

Prom RR-1
(5′ GCACGGATTCAAGTGTCGATATC 3′, SEQ ID N^{o} 12)
and
Prom RR-2
(5′ TGCGGTACTCATCAATATTTTGTATATC 3′, SEQ ID N^{o} 13)
then
Prom RR-3
(5′ AAACTCATGATAACCATGATACCTCG 3′, SEQ ID N^{o} 14)
and
Prom RR-4
(5′ CGATCTTGGAACATATAGAACAACAGTC 3′, SEQ ID N^{o} 15)

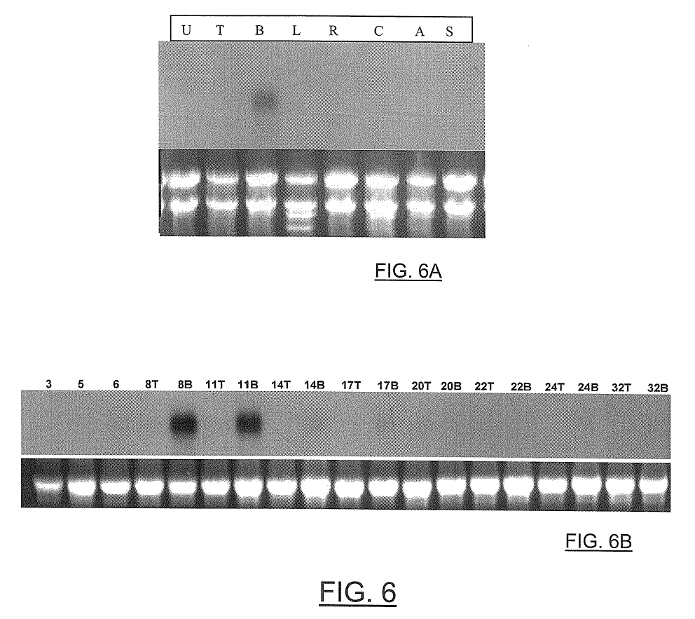

The 1.4 kb PCR product (including 200 bp of coding sequences in its termini to verify the sequence's identity) was purified and cloned in pBluescript prior to sequencing. FIG. 6 shows the final genomic sequence obtained of the putative promoter and ZmTCRR-1 coding region.

### 1.4 The Expression Pattern of ZmTCRR-1

In order to assess expression specificity, Northern Blot analyses were performed on total RNA from unpollinated female flowers, upper and lower halves of the immature seeds, leaves, roots, coleoptiles, tassel and silks. Also included in these analyses was a time series in grain development. These experiments confirmed the high specificity of ZmTCRR-1 expression to the bottom half of the seed and defined a time frame for its expression between 5 days after pollination (5 DAP) and 24 DAP, peaking around 11 DAP (FIG. 6).

### Northern Blot:

20 micrograms of total RNA from each of the following samples—unpollinated flowers, leaves, roots, coleoptiles, silks, anthers, top and bottom half of the seed, whole seed of 3, 5, 6 DAP and top and bottom halves of 8, 11, 14, 16, 20, 22, 24 and 32 DAP seeds—were electrophoresed in 1.5% agarose under denaturing conditions (6% formaldehyde) and transferred to charged nylon membranes (Roche). Blotting procedures were as described in Hueros et al, 1995. 300 bases at the 3′-end of the cDNA were labelled with ³²P for Northern Blot. Hybridisation, washing of the membranes and auto radiographic detection were performed as described in Hueros et al, 1999a.

RT-PCR experiments on the same samples, further confirmed the tissue specificity of ZmTCRR-1, as no PCR product was amplified from non-seed tissues (not shown).

### RT-PCR:

Using the sequence obtained from the library clone 3810, primers were designed in order to determine the gene's expression pattern. 500 nanograms of DNase-treated RNA obtained from unpollinated flowers, leaves, roots, coleoptiles, silks, anthers, top and bottom half of the seed was used to perform RT-PCR with the One Step RT-PCR kit (Qiagen).

### ZmTCRR-1 is Exclusively Expressed at the Transfer Cell Layer:

### In Situ Hybridisation:

Seeds of 11 and 16 DAP were fixed in paraformaldehyde/glutaraldehyde, dehydrated through an ethanol series, embedded in Fibrowax (Plano GmbH) and cut in 10 μm sections basically as described in Hueros et al, 1999b. For in situ hybridisation slides were probed with a dig-riboprobe synthesized using the Dig RNA Labelling Mix kit and SP6 RNA Polymerase (Roche) from the library plasmid containing the 3810 insert. In situ hybridisation signal was detected with NBT/BCIP (Roche) dissolved in PVA solution (10% polyvinyl alcohol, 0.1M Tris, 0.1M NaCl, 50 mM MgCl) as a substrate.

A digoxigenin-labelled antisense riboprobe was so produced from the 3810 insert and hybridised to seed sections obtained from kernels at various developmental stages. Signal was detected in the area corresponding to the basal transfer layer at 5 DAP and in the transfer cells at 11 DAP (not shown) and 16 DAP. In agreement with the results shown by the Northern analyses, signal intensity in the transfer cells reached a maximum by around 11 DAP and was hardly detectable before 5 DAP or after 16 DAP.

In these samples, accumulation of the transcript could be observed in small and immature cells located in the periphery of the transfer layer and the inner side of the tissue (not shown). No signal was detected with a sense probe used as a negative control.

### 1.5. Accumulation of the ZmTCRR-1 Protein, Sub-Cellular Localisation:

To determine the localization of the ZmTCRR-1 protein in the maize endosperm, a polyclonal antiserum was raised.

### Protein in Vitro Synthesis and Antibody Production:

The cDNA sequence of ZmTCRR-1 was cloned between the Ncol-BamHI sites of pIVEX 2.4a vector, which adds a 6× histidine tail to the N-terminal end of the protein, and used to produce the peptide in an HY 500 in vitro transcription/translation system, based on a E. coli lysate (Roche). Protein solubility and integrity was checked by Western Blot, using a primary mouse anti-His antibody (Qiagen) to detect the His-tagged protein, and a secondary antimouse antibody conjugated to horseradish peroxidase (Sigma). Detection was based on the Super Signal West Pico Chemiluminiscent Substrate (Pierce). The resulting protein was solubilized in 8M urea, affinity-purified using Ni-NTA agarose (Qiagen) and dialysed against 1M urea 0.5% SDS. Protein yield of the procedure was quantitated using the Bradford reagent (Sigma) and 4×100 microgram doses were inoculated in rabbits along an 80 days term in order to obtain a polyclonal serum.

### Immunolocalization:

Seeds of 11 and 16 DAP were fixed in paraformaldehyde/glutaraldehyde, dehydrated through an ethanol series, embedded in Fibrowax (Plano GmbH) and cut in 10 μm sections basically as described in Hueros et al, 1999b. Immunolocalization was performed using the UltraVision Detection System (Lab Vision Corporation) following the manufacturer's indications with minor modifications. PBS was used as washing buffer. Immunolocalisation signal was detected with NBT/BCIP (Roche) dissolved in PVA solution (10% polyvinyl alcohol, 0.1M Tris, 0.1M NaCl, 50 mM MgCl) as a substrate.

Immunolocalization experiments showed that the ZmTCRR-1 protein accumulates in various cell layers inwards the endosperm (not shown), rather than in the transfer cells, were the transcript is produced. At 11 DAP the protein is distributed through all the lower half of the endosperm (not shown). Interestingly, almost no signal is detected in the transfer cells (not shown). At 16 DAP (not shown), the protein can be detected even at the upper part of the endosperm, although the intensity of the signal decreases significantly. No signal was obtained with a pre-immune serum on these sections.

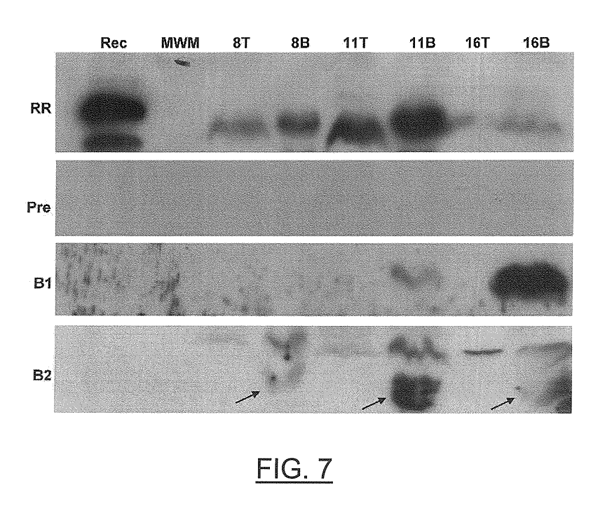

Western blot analyses of seed protein extracts further confirmed the localisation of the ZmTCRR-1 protein (FIG. 7).

### Western Blot Experiments:

The kinetics of the ZmTCRR-1 protein was assessed by western blot at three points along seed development. Kernels of 8, 11 and 16 days after pollination were excised in Top and Bottom halves, and total proteins were extracted by grinding in protein loading buffer+1 mM PMSF+1 microgram/ml leupeptin+1 microgram/ml aprotinin. Proteins were separated in 15% polyacrilamide prior to transfer to a PVDF filter (Millipore) using a modified Towbin buffer (25 mM Tris, 192 mM glycine, 20% methanol, 0.05% SDS). The filter was then subjected to immunodetection with the anti ZmTCRR-1 antiserum. The signal was detected using a chemiluminiscent substrate (Super Signal West Pico Chemiluminiscent Substrate, Pierce). Replicates of the filter were tested with preimmune serum, rabbit anti-BETL-1 (Hueros et al, 1995) and anti-BETL-2 (Serna et al, 2001) polyclonal serums to check specificity of the signal and purity of the samples, respectively.

The anti-ZmTCRR-1 antibody detected a band with the expected size in protein extracts from both upper and lower halves of the kernels at all developmental stages tested (FIG. 7, panel RR). In agreement with the results obtained in the Northern blot analyses, the protein concentration peaks at 11 DAP, especially in the bottom half, and subsequently decays at 16 DAP. No protein was detected in this area of the blot by the preinmuneserum (FIG. 7, panel Pre). The quality of the protein extracts, concerning a possible contamination of the upper-halves extracts with proteins from the lower part of the kernels, was assessed by immunoreaction of the blots with the basal kernel specific antibodies for BETL-1 (Hueros et al., 1995) and BAP-2 (Serna et al., 2001). These controls (FIG. 7, panels B1 and B2) showed that the protein extracts from the upper half of the kernels were not contaminated with basal specific proteins. This pattern of protein accumulation strongly suggests movement of the protein, possibly through plasmodesmata, from the BETL cells where it is produced towards the inner layers of endosperm cells.

### Subcellular Location of ZmTCRR-1:

The cDNA cloned in pIVEX2.4a was amplified with primers (Need primers—Probably RRfw and RR-GFP) designed to provide an amplicon with Ncol sites in both ends. This was cloned into the Ncol site of pGFP-JS (kindly provided by Dr. J. Sheen, Massachusetts General Hospital, Boston) creating a ZmTCRR-1-GFP fusion protein. Digestion with EcoRI identified a clone with the right orientation, and this was cloned uder the control of a 35S promoter and used to transform tobacco protoplasts, as described in Negrutiu et al (1987). After two days of culture at 26° C. in K3 medium (16 mM xylose, 0.5 mM Inositol, 0.4M sucrose, 1× Murashige and Skoog basal salt mixture and vitamins), protoplasts were collected, concentrated in 100 microlitres of W5 buffer (0.15M NaCl, 0.16M CaCl₂, 5 mM KCl, 5 mM glucose) and observed under UV illumination in a Zeiss Axiophot microscope. Additionally, this same construct was bombarded into onion epidermal cells using a gas-powered gun (PDU-1000/He, BioRad) following the manufacturer's instructions. Epidermis were kept for 2 days at 26° C. in the dark on solid MS medium (0.5% agarose, 100 mg/L myo-inositol, 2 g/L Asp, 2 g/L Gln, 30 g/L sucrose, MS vitamins).

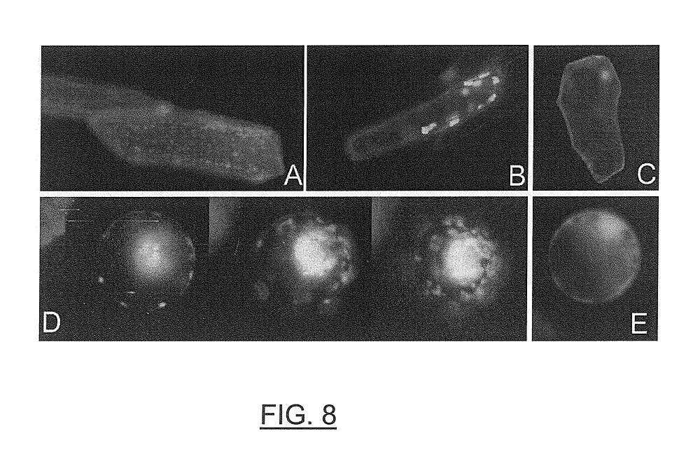

Results showed that either in tobacco protoplast (FIG. 8A, B) or in onion epithelium cells (FIG. 8D) the fusion protein was localised to the cytoplasm, forming in most cases protein aggregates that associate with membrane compartments surrounding the nuclei or close to the plasma membrane.

### Example 2

### ZmTCRR-1 Promoter Isolation

### 2.1. ZmTCRR-1 Promoter Isolation:

As described above a putative promoter sequence of ZmTCRR-1, was obtained by an inverse PCR strategy.

### 2.2. ZmTCRR-1 Promoter Transactivation by ZmMRP-1:

Southern-blot analyses using several restriction enzymes lead to the identification of HindIII as the enzyme producing the most suitable fragment for promoter isolation. Genomic DNA was digested with HindlIl and fragments between 1.6 and 2.0 kb were gel-purified, self-ligated and used as template for a PCR reaction using a proofreading enzyme (KOD Hot-Start DNA Polymerase, Novagen). The 1.4 kb PCR product (including 200 bp of coding sequences in its termini to verify the sequence's identity) was purified and cloned in pBluescript prior to sequencing.

The promoter sequence of ZmTCRR-1 presents some features resembling other transfer cell specific gene promoters. BETL-1 and 2 bear a TATC microsatellite sequence 40 to 80 bp upstream their TATA boxes (Hueros et al, 1999b). In the ZmTCRR-1 promoter, a sequence containing 5 TATC repeats is located 40 bp upstream of the putative TATA box. This fact, together with the similarities in site and time of expression found between ZmTCRR-1 and previously described transfer cell specific genes, suggests that factors controlling BETL-1 and -2 expression, namely ZmMRP-1 (Gomez et al., 2002) could also regulate ZmTCRR-1. In order to test this hypothesis, a construct containing 1200 bp of the ZmTCRR-1 promoter fused to the GUS reporter gene was assayed for transactivation by ZmMRP-1 in two transient expression systems, tobacco protoplats and onion epithelia.

### Promoter Transactivation Assays:

A 1187 bp promoter fragment isolated by I-PCR was fused to the start site of the GUS gene. This construct was used to transform onion epithelia (by particle bombardment) or tobacco protoplast (mediated by PEG), together with an Ubiquitin promoter-ZmMRP1 expression vector or an empty plasmid (PUBI-MRP and pUBI-NOS, described in Gómez et al, 2002). In the tobacco protoplast experiments, a 35s-luciferase vector was included as transformation control. In the onion epithelia system, the GUS expression signal was developed after 24 hours incubation in the dark by incubation of the epithelia in a staining solution containing X-Gluc for another 24 hours. Transformed protoplast were collected after 2 days at 26° C. of culture in K3 buffer and divided in two aliquots that were independently assayed for GUS and luciferase activity. Results, presented as GUS/Luc ratio, are the average of five replicates.

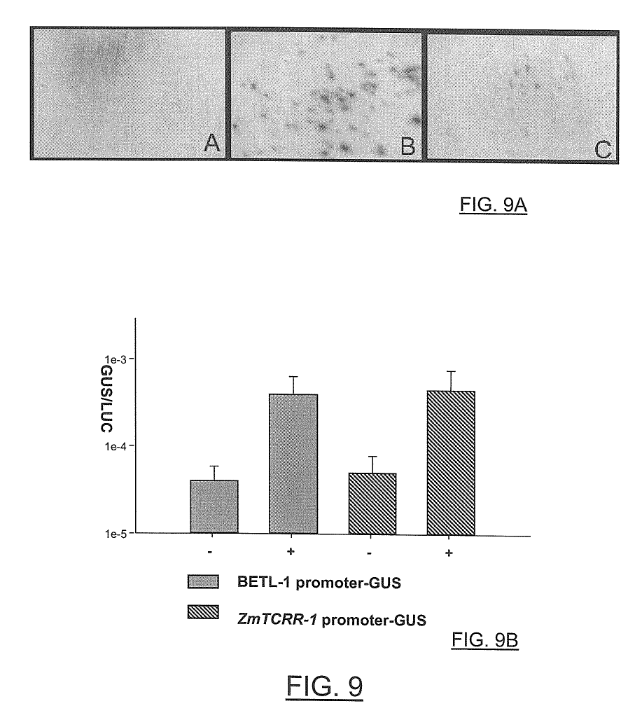

In onion epidermal cells (FIG. 9, panel 1), the reporter construct ZmTCRR-1prom-GUS was shown to be inactive in the absence of the ZmMRP-1 transcriptional activator (FIG. 9A). Co-bombardment with a construct overexpressing ZmMRP-1 under the control of the maize ubiquitin promoter (FIG. 9B), produced however a strong signal in terms of number and intensity of blue spots in the epithelia, the signal was even higher that that obtained from the Ubiquitin promoter-GUS positive control. In order to quantify the effect of ZmMRP-1 on the ZmTCRR-1 promoter, the same constructs used in the onion transient expression system were introduced in tobacco protoplasts, along with a p35s-LUC construct for transformation efficiency control. The presence of the effector plasmid expressing ZmMRP-1 under the control of the maize ubiquitin promoter increases the GUS activity driven by the ZmTCRR-1 promoter by a factor of 9.01, as compared with control experiments in which the effector plasmid was substituted by a plasmid containing the ubiquitin promoter sequence but no ZmMRP-1 (FIG. 9, panel 2, striped columns). For comparison, experiments using the BETL1promoter-GUS construct as a reporter plasmid were carried out in parallel (FIG. 9, panel 2, striped columns), as the BETL-1 promoter has been reported to be efficiently trans-activated by ZmMRP-1 in this transient expression system (Gomez et al., 2002), in this case the transactivation factor was 9.89 (FIG. 9, panel 2, solid columns). The ZmTCRR-1 promoter was thus trans-activated by ZmMRP-1 at a similar level as that obtained for the BETL-1 promoter, with negative controls for both constructs producing values of GUS activity very close to those obtained from protoplasts transformed with no reporter construct.

Since ZmMRP1 is expressed in the BETL these results support the idea that ZmTCRR-1 is expressed in the BETL and moreover suggest that a functional ZmTCRR-1 promoter region has been isolated. In the present invention, the first signal transduction element specifically expressed in the transfer cells of the maize kernel has been described and its regulation demonstrated in vivo by a transfer cell specific transcription factor, ZmMRP-1, proposed as a mediator in transfer cell development (Gómez et al., 2002).

### Example 3

### Overexpression of the ZmTCRR-1 Gene in Maize

The results in Examples 1 and 2 show that ZmTCRR-1 encodes a type A response regulator (RR) protein that in maize can be distinguished from other type A RR proteins both in the basis of sequence divergence and on the change of the conserved amino acid D to H (position 9 in ZmTCRR-1). Additionally ZmTCRR-1 has a unique expression and protein localisation pattern in that the gene is expressed in the BETL but the protein is localised in the starchy endosperm layer, a most unexpected result.

Response regulators are known to be components of the “two-component system” or “phosphorelay system” signal transduction pathways. These systems have been found to be part of the sensory arsenal of plants like Arabidopsis, whose genome harbours about forty genes with sequence similarity to phosphorelay-type genes (D'Agostino et al., 1999;) and maize (Yamada et al., 1998; Takei et al., 2002). In Arabidopsis the implication of two-component systems in cytokinin and ethylene mediated signalling is being intensively studied (Taniguchi et al., 1998; Lohrmann et al., 2002). Response regulators have been divided in two classes, which in plant are identified as type-A (small size, short or nonexistent C-terminal domain, cytokinin inducible, unknown function) and type-B (longer C-terminal extension with similarity to Myb-related DNA binding domains, not inducible by cytokinin, probably involved in transcriptional regulation). Currently known type-A response regulators are controlled by cytokinin, which has in at least one case been shown to exert its effect through a type-B molecule. Sakai et al. (2001) showed the expression of ARR6 mRNA is controlled by ARR1, which mediates cytokinin signals. Interestingly cytokinin levels during grain filling are known to be positively correlated with the extent of cell division in the endosperm during the lag phase of endoserm development. Importantly the number of cells in the endosperm is positively correlated with potential grain size and yield. The expression pattern of ZmTCRR-1 also correlates with the level of cell division in the endosperm with a peak around 11 DAP and a subsequent rapid fall when cell division ceases in the endosperm and grain filling commences with the accumulation of storage products such as starch. Thus a plausible role for ZmTCRR-1 is the transmission or modulation of the cytokinin signal in the endosperm perhaps sensing the state of development or activity of the BETL layer. Changing ZmTCRR-1 levels or activity would thus influence endosperm cell division and potential grain yield. In support of this hypothesis the chromosomal position of ZmTCRR-1 co-localises with several QTLs for grain yield.

Thus Overexpression of ZmTCRR-1 in the endosperm is predicted to stimulate endosperm cellularization, cell division and differentiation independantly of cytokinin levels. This overexpression can increase the number of endosperm divisions in the lag phase of development or extend the period of the lag phase giving an endosperm with more cells. Endosperms with more cells will potentially give seed with higher weights and yield. Thus ZmTCRR-1 was overexpressed in the seed either by using a consititutive promoter or endosperm specific promoters. Suitable endosperm specific promoters are for example BETL promoters such as pBETL, pBETL2 and the promoter of ZmTCRR-1.

### 3.1. Constructs Preparation

### Example 3.1.1

### Constitutive Overexpression of ZmTCRR-1

The coding region of ZmTCRR-1 was amplified using primers that contained aatB1 or aatB2 sites and the product recombined into the pDONR221 vector (Invitrogen) using a BP recombinase reaction. In the resulting GATEWAY ENTR clone, named pDONR221/ZmRR the 5′ region of ZmTCRR-1 is adjacent to the attL1 site. The ZmTCRR-1 coding region was then placed under the control of the constitutive pCsVMV promoter (Verdaguer et al. (1996)) by performing an LR recombination reaction with the GATEWAY destination binary vector pBIOS 886 forming pBIOS951. The vector pBIOS 886 is a derivative of pSB12 (Komari et al. (1996)) containing a marker gene under the pActin promoter for selection of maize transformants, a pCsVMV-GFP gene to follow the presence of the transgene in plants and seeds and a CsVMV promoter linked to an actin intron (McElroy et al. (1990)) followed by a GATEWAY cassette and a polyadenylation sequence derived from the Arabidopsis Sac66 gene (Jenkins et al. (1999)).

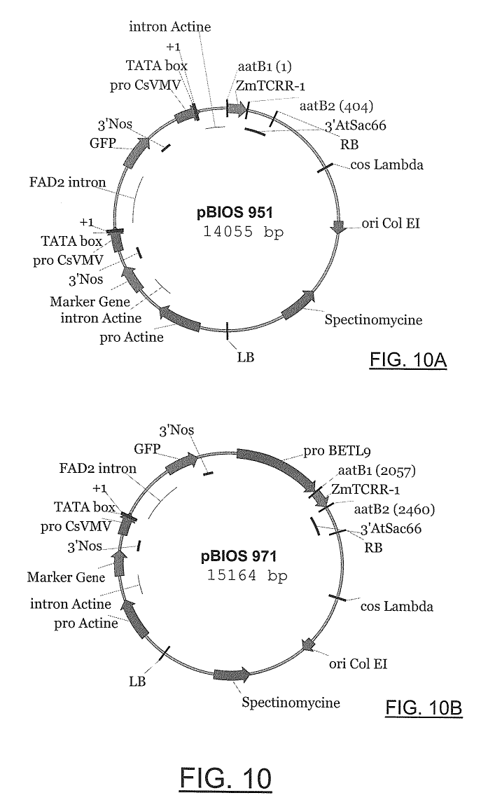

pBIOS 951 (FIG. 10A) was transferred into agrobacteria LBA4404 (pSB1) according to Komari et al. (1996) and the Maize cultivar A188 was transformed with this agrobacterial strain essentially as described by Ishida et al. (1996).

The transformed plants overexpressing ZmTCRR-1 possess a normal vegetative phenotype however seed inheriting the transgene have a larger size and weight compared to seed on the same plants that lack the transgene.

### Example 3.1.2

### Overexpression of ZmTCRR-1 in the BETL

The coding region of ZmTCRR-1 was amplified using primers that contained aatB1 or aatB2 sites and the product recombined into the pDONR221 vector (Invitrogen) using a BP recombinase reaction. In the resulting GATEWAY ENTR clone, named pDONR221/ZmRR, the 5′ region of ZmTCRR-1 is adjacent to the attL1 site. The ZmTCRR-1 coding region was then placed under the control of the BETL-specific pBETL9 maize promoter by performing an LR recombination reaction with the GATEWAY destination binary vector pBIOS 960 forming pBIOS 971. The vector pBIOS 960 is a derivative of pSB12 (Komari et al. (1996)) containing a marker gene under the pActin promoter for selection of maize transformants, a pCsVMV-GFP gene to follow the presence of the transgene in plants and seeds and a BETL9 promoter followed by a GATEWAY cassette and a polyadenylation sequence derived from the Arabidopsis Sac66 gene (Jenkins et al. (1999)). The BETL9 gene has an expression pattern similar to the BETL1 gene (Hueros et al, (1995)). The 1941 bp maize BETL9 promoter was PCRed from genomic DNA of the inbred line F2 using the primers:

pBETL9fw

(SEQ ID N^{o} 16)

5′ CGATGGTACTTACTCATGATGGTCATCTAGG 3′,
and
pBETL9rw

(SEQ ID N^{o} 17)

5′ CCATGGTATAACTTCAACTGTTGACGG 3′,.

pBIOS 971 (FIG. 10B) was transferred into agrobacteria LBA4404 (pSB1) according to Komari et al. (1996) and the Maize cultivar A188 was transformed with this agrobacterial strain essentially as described by Ishida et al. (1996).

The transformed plants overexpressing ZmTCRR-1 possess a normal vegetative phenotype however seed inheriting the transgene have a larger size and weight compared to seed on the same cob that lack the transgene.

A fragment of the maize BETL9 promoter (1911 bp) led to the same results. This fragment is represented by SEQ ID N^{o} 18.

### 3.2. Effect of ZmTCRR-1 Gene on Seed Size and Weight

In order to measure the effect of ZmTCRR-1 gene on seed size and/or seed weight, transgenic (T) and non-transgenic (WT) maize seeds have been compared.

Transgenic maize plants have been produced according to example 3.1.2, and thus overexpress the ZmTCRR-1 gene under the control of the pBETL9 promoter (pBIOS 971 vector). To achieve these comparisons, twenty transgenic and twenty non-transgenic kernels have been randomly taken from a maize ear of the m60C1 plant transformation event. GFP has been used to distinguish transgenic and non-transgenic maize kernels.

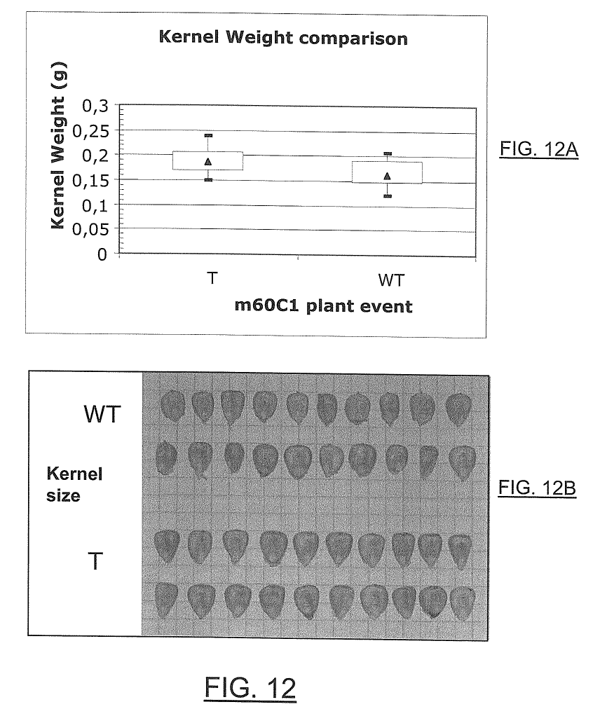

FIG. 12A shows that transgenic (T) maize kernels have and increased weight when compared to non-transgenic (WT) kernels.

TABLE 1

| | | | | WT | | Average |
|---|---|---|---|---|---|---|
| Plant | T kernel | Number | Average | kernel | Number | kernel |
| event | weight | of kernels | kernel | weight | of kernels | weight |
| code | (g) | analyzed | weight (g) | (g) | analyzed | (g) |
| m60C1 | 3.861 | 20 | 0.19305 | 3.389 | 20 | 0.16945 |

Table 1 shows that kernel weight is increased by about 14%.

FIG. 12B shows that transgenic (T) maize kernels have an increased size when compared to non-transgenic (WT) kernels.

### Example 4

### Repression of ZmTCRR1

### Example 4.1

### Repression of ZmTCRR-1 Expression Using the Constitutive pCsVMV Promoter

The coding region of ZmTCRR-1 was amplified using primers that contained aatB1 or aatB2 sites and the product recombined into the pDONR221 vector (Invitrogen) using a BP recombinase reaction. In the resulting GATEWAY ENTR clone, named pDONR221/ZmRR, the 5′ region of ZmTCRR-1 is adjacent to the attL1 site. The ZmTCRR-1 coding region was then placed under the control of the constitutive pCsVMV promoter (Verdaguer et al. (1996)) in an inverted repeat orientation by performing an LR recombination reaction with the GATEWAY destination RNAi binary vector pBIOS 893 forming pBIOS 946. The vector pBIOS 893 is a derivative of pSB12 (Komari et al. (1996)) containing a marker gene under the pActin promoter for selection of maize transformants, a pCsVMV-GFP gene to follow the presence of the transgene in plants and seeds and a CsVMV promoter linked to an actin intron (McElroy et al. (1990)) followed by two GATEWAY cassettes in opposite orientations separated by a rice tubulin intron. This GATEWAY RNAi region is followed by a polyadenylation sequence derived from the Arabidopsis Sac66 gene (Jenkins et al. (1999)).

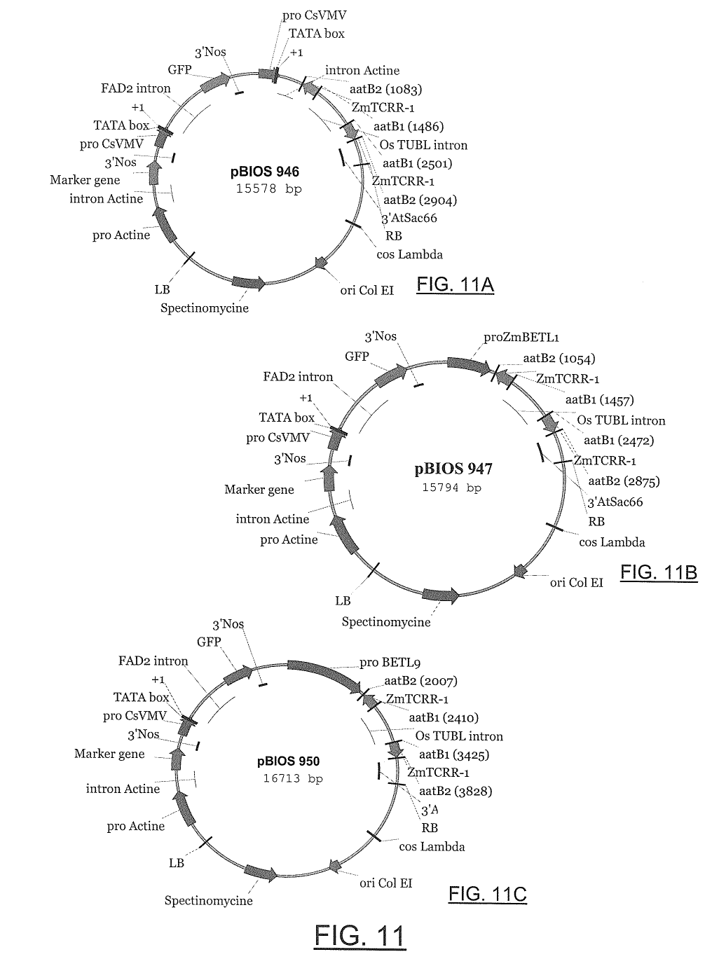

pBIOS 946 (FIG. 11A) was transferred into agrobacteria LBA4404 (pSB1) according to Komari et al. (1996) and the Maize cultivar A188 was transformed with this agrobacterial strain essentially as described by Ishida et al. (1996).

The transformed plants with reduced ZmTCRR-1 expression possess a normal vegetative phenotype however seed inheriting the transgene have a smaller size and weight compared to seed on the same cob that lack the transgene.

### Example 4.2

### Repression of ZmTCRR-1 Expression Using the BETL-Specific BETL1 Promoter

The coding region of ZmTCRR-1 was amplified using primers that contained aatB1 or aatB2 sites and the product recombined into the pDONR221 vector (Invitrogen) using a BP recombinase reaction. In the resulting GATEWAY ENTR clone, named pDONR221/ZmRR, the 5′ region of ZmTCRR-1 is adjacent to the attL1 site. The ZmTCRR-1 coding region was then placed under the control of the BETL-specific pBETL1 maize promoter (Hueros et al (1999a)) in an inverted repeat orientation by performing an LR recombination reaction with the GATEWAY destination RNAi binary vector pBIOS 942 forming pBIOS 947. The vector pBIOS 942 is a derivative of pSB12 (Komari et al. (1996)) containing a marker gene under the pActin promoter for selection of maize transformants, a pCsVMV-GFP gene to follow the presence of the transgene in plants and seeds and a BETL1 promoter followed by two GATEWAY cassettes in opposite orientations separated by a rice tubulin intron. This GATEWAY RNAi region is followed by a polyadenylation sequence derived from the Arabidopsis Sac66 gene (Jenkins et al. (1999)).

pBIOS 947 (FIG. 11B) was transferred into agrobacteria LBA4404 (pSB1) according to Komari et al. (1996) and the Maize cultivar A188 was transformed with this agrobacterial strain essentially as described by Ishida et al. (1996).

The transformed plants with reduced ZmTCRR-1 expression possess a normal vegetative phenotype however seed inheriting the transgene have a smaller size and weight compared to seed on the same cob that lack the transgene.

### Example 4.3

### Repression of ZmTCR-1 Expression Using the BETL-Specific BETL9 Promoter

The coding region of ZmTCRR-1 was amplified using primers that contained aatB1 or aatB2 sites (primers seqs) and the product recombined into the pDONR221 vector (Invitrogen) using a BP recombinase reaction. In the resulting GATEWAY ENTR clone, named pDONR221/ZmRR, the 5′ region of ZmTCRR-1 is adjacent to the attL1 site. The ZmTCRR-1 coding region was then placed under the control of the BETL-specific pBETL9 maize promoter in an inverted repeat orientation by performing an LR recombination reaction with the GATEWAY destination RNAi binary vector pBIOS 945 forming pBIOS 946. The vector pBIOS 945 is a derivative of pSB12 (Komari et al. (1996)) containing a pActin-Bar gene for selection of maize transformants, a pCsVMV-GFP gene to follow the presence of the transgene in plants and seeds and a BETL9 promoter followed by twoGATEWAY cassettes in opposite orientations separated by a rice tubulin intron. This GATEWAY RNAi region is followed by a polyadenylation sequence derived from the Arabidopsis Sac66 gene (Jenkins et al. (1999)). The BETL9 gene is homologous to the barley END1 gene Daon et al (1996) and has an expression pattern similar to the BETL1 gene (Hueros et al, (1995)). The 1941 bp maize BETL9 promoter was PCRed from genomic DNA of the inbred line F2 using the primers:

pBETL9fw

(SEQ ID N^{o} 16)

5′ CGATGGTACTTACTCATGATGGTCATCTAGG 3′
and
pBETL9rw

(SEQ ID N^{o} 17)

5′ CCATGGTATAACTTCAACTGTTGACGG 3′.

pBIOS 950 (FIG. 11C) was transferred into agrobacteria LBA4404 (pSB1) according to Komari et al. (1996) and the Maize cultivar A188 was transformed with this agrobacterial strain essentially as described by Ishida et al. (1996).

The transformed plants with reduced ZmTCRR-1 expression possess a normal vegetative phenotype however seed inheriting the transgene have a smaller size and weight compared to seed on the same cob that lack the transgene.

A fragment of the maize BETL9 promoter (1911 bp) led to the same results. This fragment is represented by SEQ ID N^{o} 18.

### Example 5

### Additional Comments on the Results Obtained

It has been described a maize response regulator, ZmTCRR-1 (plant signal transduction protein), specifically expressed in a very discrete region of the kernel, the basal transfer layer. ZmTCRR-1 was isolated as a moderately expressed transcript in the lower half of the seed.

The full-length cDNA is 667 bp long and shows no significant homology with EST sequences in the databases. However, when BLASTx is used, a relevant similarity is found at the protein level with transcripts for response regulators in maize and rice, showing high conservation in functionally critical domains. A pair-wise sequence comparison of ZmRRs 1-7, all of them type-A molecules (Asakura et al., 2003), to ZmTCRR-1 produces identities between 21.1% and 42.6%. When the whole group is considered, identity descends to 12.5%, except in regions highly conserved in type-A response regulators (Hwang et al., 2002).

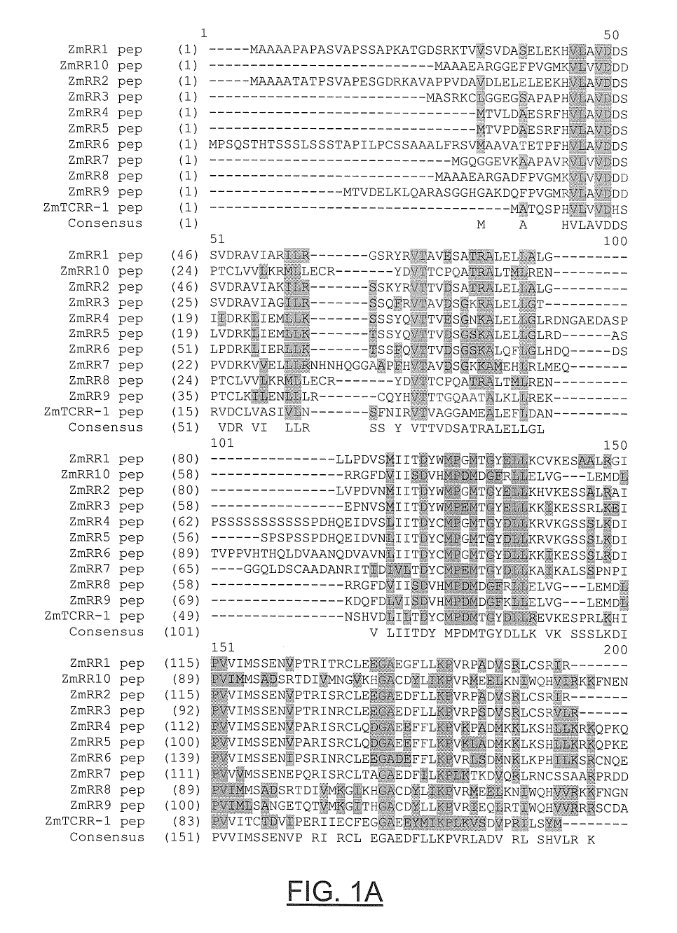

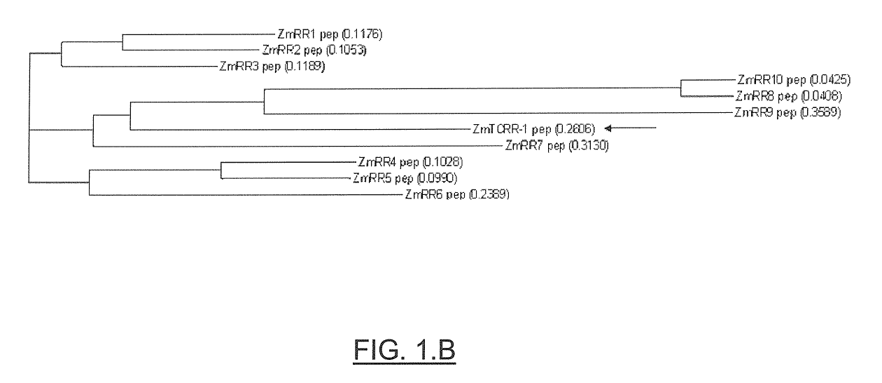

The protein encoded by ZmTCRR-1 (FIG. 1) is small in size (124aa, 13.8 kD), and acid (pl 4.79), no secretion domain can be predicted in its structure. Almost all the protein is included in the response regulator domain, with a very short C-terminal extension. This strongly suggests that the motif itself is responsible for the protein's function, instead of regulating an adjacent domain as is usually the case in other type-A RRs in plants, with C-terminal domains ranging from 30 to 100 aminoacids (D'Agostino and Kieber, 1999).

ZmTCRR-1 shows the conserved residues archetypical of type-A RRs with one exception: Aspartic 13 is substituted by a histidine.

The expression pattern of ZmTCRR-1 is restricted to the transfer cell layer at the base of the kernel, no transcript could be detected in any other plant tissue tested. During kernel development, the transcript accumulate at the base of the seeds in a very narrow time-window, between 10 and 14 DAP. Significantly, transfer cells are actively differentiating at these stages (Hueros et al., 1999b). Cell walls proliferate to transform the cubic cells found at the base of the endosperm by 5-8 DAP into the elongated, cell wall ingrowth-filled cells encountered at 16 DAP. The differentiation process also progresses spatially, as monitored by the development of cell wall ingrowths. At 11 DAP, the most differentiated cells are encountered in a basal area near the germinal pole, whilst cells at the abgerminal side or at the inner layers have few or no cell wall ingrowths. In situ hybridisation results show that the ZmTCRR-1 transcript accumulation follows the transfer cell differentiation process described above, the transcript accumulates preferentially at the immature basal cells positioned at both edges of the transfer cell layer.

Similarly to other BETL genes, ZmTCRR-1 accumulates out of the cells where it is transcribed. However, in opposition to other BETL proteins, the protein is exported into the endosperm and not to the maternal tissues. The ZmTCRR-1 protein seems to accumulate in the lower half of the endosperm, and is absent in the transfer layer, where no signal can be detected after 11 DAP. ZmTCRR-1 function being connected to signal transduction pathways, it might be involved in some of the important processes taking place in the endosperm at this stage, which include end of cell division and start of nutrient accumulation (Young and Gallie, 2000). Localisation of the ZmTCRR-1 protein in the inner layers of the endosperm was a very unexpected result, and the inventors tried therefore to confirm the immunolocalisation results by western-blot analyses, the results of these experiments fully confirmed the observations at the microscope. The ZmTCRR-1 protein is present in protein extracts from the upper half of the kernels, which completely lacked other proteins derived from transfer cell specific genes.

Most BETL proteins show putative signal peptides in the N-terminal end of their molecules. The absence of known signal peptides in the sequence of ZmTCRR-1 suggests that protein movement is not mediated by secretion. It is strongly suggested that the protein translocates through a symplastic pathway, via plasmodesmata. Interestingly, when tobacco protoplasts and onion epidermal cells were transformed with a ZmTCRR-1-GFP fusion, signal was concentrated in clumps associated to the membranous system either at the perinuclear or plasma membrane locations. This association to the membranous system further supports the existence of a symplastic transport mechanism.

As no transmembrane domains could be found in the ZmTCRR-1 sequence, membrane association may be due to post-translational modifications or interactions with membrane proteins. Pfam analysis shows a putative myristylation site in the structure, which could link the protein to the membrane.

However, the coincidence in site and timing of expression between ZmTCRR-1 and some BETL genes made the former a good candidate for being expressed under the control of ZmMRP-1, another single domain Myb-related transcriptional regulator. ZmMRP-1 controls the expression of BETL1 and 2, as shown in protoplast cotransformation experiments (Gomez et al., 2002), and it contains the SHAQKYF sequence in its DNA-binding domain, which resembles the B-motif (Hosoda et al., 2002). Several additional facts further support the hypothesis that ZmMRP1 is at least partially responsible for the control of ZmTCRR-1 in vivo. First, the coincidence in site and timing of expression between ZmMRP-1 and ZmTCRR-1. The kinetics of ZmTCRR-1 mRNA correlates well with that of ZmMRP-1, with a small delay for the expression of ZmTCRR-1, as expected if this gene was indeed regulated by ZmMRP-1 (Carey and Smale, 2000). Secondly, the promoter region of ZmTCRR-1 displays a 48 bp tract with 5 TATC repeats in region −184 to −136, a position and sequence structure very similar to those ones existing in the BETL-1 and -2 promoters (Gomez et al., 2002; Hueros et al., 1999b). Thirdly, the transactivation levels shown in protoplast cotransformation assays for ZmMRP-1 and ZmTCRR-1 promoter (FIG. 7) are roughly similar to those found for the BETL-1 promoter, which is a biological target for ZmMRP-1 (Gomez et al., 2002; Hueros et al., 1999a).

Transfer cells seem to be involved in critical processes for the life cycle of the plant, such as nutrient exchange and the establishment of defensive barriers against pathogens. In the present invention, it has been described the first signal transduction element specifically expressed in the transfer cells of the maize kernel and demonstrated its regulation in vivo by a transfer cell specific transcription factor, ZmMRP-1, proposed as a mediator in transfer cell development (Gómez et al., 2002).

Cell to cell movement and possible long lasting activation caused by the D-H substitution suggest a working model for the function of ZmTCRR-1 in which the protein is constitutively produced at the basal layer of transfer cells, once activated by a signal or signals originated at the maternal side of the kernel, the activated ZmTCRR-1 would migrate, and transport the signal, to the inner layers of the endosperm. Localisation of the ZmTCRR-1 protein in the cells connecting transfer cell layer and endosperm crown, a tissue some times referred to as conducting tissue (Becraft, 2001), strongly suggests a role for this gene in its differentiation. This tissue is believed to provide a constant flux of metabolites for storage product synthesis.

### REFERENCES

Asakura Y., et al. (2003) Plant Molecular Biology 52, 331-341.
Becraft, P. W. (2001) Cell fate specification in the cereal endosperm. Semin Cell Dev Biol. 12(5):387-94.
Bradford M. M. Analytical Biochemistry 72, 248-254. 1976.
Brandstatter I., Kieber J. J. The Plant Cell 10, 1009-1019. 1998.
Bourret R. B. et al., PNAS 87, 41-45. 1990.
Carey M., Smale S. T. Transcriptional regulation in eucaryotes. Chapter 9, 291-317. Ed. Cold Spring Harbor Laboratory Press. 2000.
Cho H. S., et al. Current Opinion in Structural Biology 11, 679-684. 2001.
Daon D N P, Linnestad C and Olsen O-A. (1996). Plant Mol. Biol 31, 877-866.
D'Agostino I. B., Kieber J. J. Phosphorelay signal transduction: the emerging family of plant response regulators. TIBS 24, 452-456. 1999.
D'Agostino I. B. et al. Plant Physiology 124, 1706-1717. 2000.
Deji A., et al. Biochimica et Biophysica Acta 1492, 216-220. 2000.
Egan S. M., Stewart V. Journal of Bacteriology 173, 4424-32. 1991.
Emanuelsson O., et al. J. Mol. Biol. 300, 1005-1016, 2000.
Gawronska H., et al. Plant Physiology and Biochemistry 41, 605-610. 2003.
Gómez E., Royo J., Guo Y., Thompson R., Hueros G., The Plant Cell 14, 598-610. 2002.
Hwang I., Chen H-C., Sheen J., Plant Physiology 129, 500-515. 2002.
Hofmann K., Stoffel W. TMbase—A database of membrane spanning proteins segments. Biol. Chem. Hoppe-Seyler 374, 166. 1993.
Hosoda K., et al. The Plant Cell 14, 2015-2029. 2002.
Hueros G., Varotto S., Salamini F., Thompson R. D. The Plant Cell 7, 747-757. 1995.
Hueros G., Gomez E., Cheikh N., Edwards J., Weldon M., Salamini F., Thompson R. D. Plant Physiology 121, 1143-1152. 1999a.
Hueros G., Royo J., Maitz M., Salamini F., Thompson R. D. Plant Molecular Biology 41 (3), 403-414. 1999b.
Hueros G et al. Plant Journal October 2006;48(1):17-27. Epub Aug. 22, 2006.
Imamura A., Hanaki N., Umeda H., Nakamura A., Suzuki T., Ueguchi Ch., Mizuno T. PNAS 95, 2691-2696. 1998.
Imlaut A., Truernaut E., Sauer N. The Plant Cell 11, 309-322. 1999.
Ishida et al., (1996), Nature Biotechnology, vol. 14: 745-750.
Jackson D. et al., The Plant Cell 13, 2569-2572. 2001.
Jenkins et al., (1999). Plant Cell and Environment 22,159-167.
Kakimoto T. Science 274(5289), 982-985. 1996.
Kenney L. J. Current Opinion in Microbiology 5, 135-141. 2002.
Kiesselbach T. A. The structure and reproduction of corn. Cold Spring Harbor Laboratory Press, Ed. 1949.
Komari T. et al., Plant J. July 1996;10(1):165-74.
Lohrmann J., Harter K. Plant Two-Component Signaling Systems and the Role of
Response Regulators. Plant Physiology 128, 363-369. 2002.
Lucas W. J., Bouche-Pillon S., Jackson D. P., Nguyen L., Baker L., Ding B. Hake S., Science 270, 1980-1983. 1995.
Maeda T., Wurgler-Murphy S. M., Saito H. Nature 369, 242-245. 1994.
Maniatis T., Fritsch E., Sambrook J. Molecular Cloning: A Laboratory Manual. Cold Spring Harbor Press, 1982.
McElroy et al. (1990), Plant Cell, 2:163-171.
Negrutiu I., Shillito R., Potrykus I., Biasini G., Sala F. Plant Mol. Biol. 8, 363-373. 1987.
Oka A., Sakai H., Iwakoshi S. Genes Genet. Syst. 77, 383-391. 2002.
Parkinson, J. S., Kofoid E. C. Communication modules in bacterial signaling proteins. Annual Review of Genetics 26, 71-112. 1992.
Patrick J. W, Offler C. E. Journal of Experimental Botany 52(356), 551-564. 2001.
Sakai H., Honma T., Aoyama T., Sato S., Kato T., Tabata S., Oka A. Science 294(5546), 1519-1521. 2001.
Sakakibara H., Suzuki M., Takei K., Deji A., Taniguchi M., Sugiyama T. The Plant Journal 14(3), 337-344. 1998.
Sakakibara H., et al. Plant Molecular Biology 42, 273-278. 2000.
Serna A., et al The Plant Journal. 25(6), 687-698. 2001.
Stock A. M., Robinson V. L., Goudreau P. N. Annual Review of Biochemistry 69, 183-215. 2000.
Szurmant H., et al. J. Biol. Chem 278(49), 48611-6. 2003.
Takei K., Takahashi T., Sugiyama T., Yamaya T., Sakakibara H. Journal of Experimental Botany 53, 971-977. 2002.
Taniguchi M., Kiba T., Sakakibara H., Ueguchi Ch, Mizuno T., Sugiyama T. FEBS Letters 429, 259-262. 1998.
Thompson R. D., Hueros G., Becker H-A., Maitz M. Plant Science 160, 775-783. 2001.
Verdaguer et al., Plant Mol Biol. September 1996;31(6):1129-39.
White S. E., Doebley J. F. Genetics 153, 1455-1462. 1999.
Yamada H., Hanaki N., Imamura A., Ueguchi Ch., Mizuno T. FEBS letters 436, 76-80. 1998.
Young T. E., Gallie D. R. Plant Molecular Biology 44, 283-301. 2000.
Bolchi, A., Petrucco, S., Tenca, P. L., Foroni, C. and Ottonello, S. (1999) Plant Mol. Biol. 39 (3), 527-537.
Depicker et al. (1992), Mol. Gen. Genet., 235(2-3):389-396
Franck et al. (1980), Cell, 21(1):285-94
Jang et al., (1997) Plant Cell, 9, 5-19
Poole et al., (1985), Cell, 40:37-43
Lobreaux S. Massenet O. and J. F. Briat (1992). Plant Molecular Biology 19: 563-575.
Zhang et al. (1995). Planta, 196:84-94.
Mary E. Knight et al. (1998), The Plant Journal 14(5), 613-622.
Ming Gao et al. (1997), Plant Physiol 114:69-78.
Taliercio E W, Kim J-Y, Mahe A., Shanker S., Choi J., Cheng W-H, Prioul J-L and P. S. Chourey, (1999). J. Plant Physiol. Vol. 155 pp. 197-204.
Thomas W. Greene and L. Curtis Hannah (1998), The Plant Cell, Vol. 1295-1306.

1. An isolated nucleic acid molecule encoding a plant signal transduction protein, comprising a sequence selected from the group consisting of:
a) a nucleotide sequence encoding a protein having the amino acid sequence represented by SEQ ID No: 2;
b) the nucleotide sequence represented by SEQ ID No: 1;
c) a sequence hybridizing under stringent conditions with the complementary strand of a nucleic acid molecule as defined in (a) or (b), and coding for a protein having signal transduction activity.

2. The isolated nucleic acid molecule according to claim 1, which has been isolated from maize.

3. The isolated nucleic acid molecule according to claim 2, wherein the sequence hybridizing under stringent conditions with the complementary strand of a nucleic acid molecule as defined in (a) or (b), and coding for a protein having signal transduction activity is an allelic variant of SEQ ID No: 1.

4. An expression cassette comprising a nucleic acid molecule according to claim 1 operatively linked to regulatory elements allowing the expression in prokaryotic and/or eukaryotic host cells.

5. The expression cassette according to claim 4 which further comprises a selection marker gene for plants.

6. An expression vector containing at least an expression cassette according to claim 4.

7. A host cell containing at least a vector according to claim 6.

8. A transgenic plant or a part of a transgenic plant, comprising stably integrated into its genome a nucleic acid molecule of claim 1, operatively linked to regulatory elements allowing transcription and/or expression of the nucleic acid molecule in plant cells.

9. The plant or part of a plant according to claim 8, wherein said plant or part of plant is a cereal or oily plant.

10. The plant or part of a plant according to claim 9, wherein said plant is selected from the group consisting of maize, rice, wheat, barley, rape, and sunflower.

11. A plant signal transduction protein encoded by a nucleic acid molecule of claim 1.

12. A method of obtaining a plant having improved agronomic qualities, comprising the steps consisting of:
a) transforming at least a plant cell by means of at least a vector according to claim 6;
b) cultivating the cell(s) thus transformed so as to generate a plant containing in its genome at least an expression cassette from said vector, whereby said plant has improved agronomic qualities.

13. A method according to claim 12, wherein said plant is maize plant.
